(19) United States
(12) Patent Application Publication
(10) Pub. No.: US 2022/0205017 A1
(43) Pub. Date: Jun. 30, 2022

(54) METHODS AND COMPOSITIONS FOR ENHANCED GENOME COVERAGE AND PRESERVATION OF SPATIAL PROXIMAL CONTIGUITY

(71) Applicant: ARIMA GENOMICS, INC., San Diego, CA (US)

(72) Inventors: Anthony SCHMITT, Apex, NC (US); Siddarth SELVARAJ, San Marcos, CA (US); Bret REID, San Diego, CA (US); Stephen MAC, Baldwin Park, CA (US); Xiang ZHOU, San Diego, CA (US)

(21) Appl. No.: 17/610,414

(22) PCT Filed: May 19, 2020
(86) PCT No.: PCT/US2020/033666

§ 371 Date: Nov. 10, 2021

Related U.S. Application Data
(60) Provisional application No. 62/850,449, filed on May 20, 2019.

(51) Int. Cl. C12Q 1/6806; C12Q 1/6874; C12N 15/10

(52) U.S. Cl. CPC C12Q 1/6806; C12N 15/1093; C12Q 1/6874

(57) ABSTRACT

Provided herein are methods and compositions for preparing sequencing templates that provide uniform genome coverage and preserve spatial-proximal contiguity information.

### RELATED PATENT APPLICATION(S)

This application is a 35 U.S.C. 371 national phase application of International Patent Cooperation Treaty (PCT) Application No. PCT/US2020/033666, filed on May 19, 2020, entitled METHODS AND COMPOSITIONS FOR ENHANCED GENOME COVERAGE AND PRESERVATION OF SPATIAL PROXIMAL CONTIGUITY, naming Anthony SCHMITT et al. as inventors, and designated by attorney docket no. AMG-1004-PC, which claims the benefit of U.S. Provisional Patent Application No. 62/850,449 filed May 20, 2019 entitled METHODS AND COMPOSITIONS FOR ENHANCED GENOME COVERAGE, naming Anthony Schmitt, Bret Reid, Stephen Mac, Xiang Zhou and Siddarth Selvaraj as inventors and assigned attorney docket no. AMG-1004-PV. This application is related to U.S. application Ser. No. 16/689,002 filed Nov. 19, 2019 entitled METHODS FOR PREPARING NUCLEIC ACIDS THAT PRESERVE SPATIAL-PROXIMAL CONTIGUITY INFORMATION, naming Anthony Schmitt, Catherine Tan, Derek Reid, Chris De La Torre and Siddarth Selvaraj as inventors and assigned attorney docket no. AMG-1003-UT. This application is also related to U.S. application Ser. No. 16/764,787 filed May 15, 2020 entitled PRESERVING SPATIAL-PROXIMAL CONTIGUITY AND MOLECULAR CONTIGUITY IN NUCLEIC ACID TEMPLATES, naming Siddarth Selvaraj, Anthony Schmitt and Bret Reid as inventors and assigned attorney docket no. AMG-1002-US. This application is also related to U.S. application Ser. No. 15/738,871 filed Dec. 21, 2017, entitled ACCURATE MOLECULAR DECONVOLUTION OF MIXTURE SAMPLES, naming Siddarth Selvaraj, Nathaniel Heintzman and Christian Edgar Laing as inventors and assigned attorney docket no. AMG-1001-US. The entire content of the foregoing patent applications are incorporated herein by reference, including all text, tables and drawings.

### STATEMENT OF GOVERNMENT SUPPORT

This invention was made with government support under Contract Nos. 1R44HG009584-01 and 2R44HG008118-04A1 awarded by the National Institutes of Health. The government has certain rights in the invention.

### FIELD

The technology relates in part to sequencing nucleic acids.

### BACKGROUND

Next-generation sequencing (NGS) has emerged as the predominant set of methods for determining nucleic acid sequence for a plethora of research and clinical applications. The typical NGS workflow is as follows: the native genomic DNA, often organized as chromosome(s), is isolated from the nucleic acid source leading to its fragmentation, to produce nucleic acid templates which are subsequently read by a sequencing instrument to generate sequence data.

### SUMMARY

The technology pertains to methods for preparing DNA molecules in such a way that preserves spatial-proximal contiguity information and provides full genome coverage equivalent to the coverage of whole genome sequencing.

Provided in certain aspects is a method for preparing DNA molecules from a sample comprising: (a) contacting cross-linked DNA molecules of a sample comprising a genome or portion thereof with a set of restriction endonucleases; thereby generating spatial-proximal digested ends of cross-linked DNA molecules; (b) contacting the spatial-proximal digested ends of cross-linked DNA molecules with ligase, thereby generating cross-linked proximity-ligated DNA molecules comprising ligation junctions; (c) contacting the cross-linked proximity-ligated DNA molecules comprising ligation junctions with a reagent that reverses cross-linking, thereby generating proximity-ligated DNA molecules comprising ligation junctions; and (d) fragmenting the proximity-ligated DNA molecules to generate fragments of proximity-ligated DNA molecules comprising fragments spanning the ligation junctions, wherein fragments spanning the ligation junctions and of lengths that can be templates for short range sequencing, comprise sequences of essentially the whole genome or portion thereof.

Also provided in certain aspects is a method for preparing DNA molecules from a sample comprising (a) contacting cross-linked DNA molecules of a sample comprising a genome or portion thereof with a first restriction endonuclease, thereby generating first spatial-proximal digested ends of cross-linked DNA molecules; (b) contacting the first spatial-proximal digested ends of cross-linked DNA molecules with ligase, thereby generating first cross-linked proximity-ligated DNA molecules comprising first ligation junctions; (c) contacting the first cross-linked proximity-ligated DNA molecules comprising first ligation junctions with a second restriction endonuclease, thereby generating second spatial-proximal digested ends of cross-linked DNA molecules; (d) contacting the second spatial-proximal digested ends of cross-linked DNA molecules with ligase, thereby generating second cross-linked proximity-ligated DNA molecules comprising first and second ligation junctions; (d) contacting the second spatial-proximal digested ends of cross-linked DNA molecules with ligase, thereby generating second cross-linked proximity-ligated DNA molecules comprising first and second ligation junctions; (e) contacting the second cross-linked proximity-ligated DNA molecules comprising first and second ligation junctions with a third restriction endonuclease, thereby generating third spatial-proximal digested ends of cross-linked DNA molecules; (f) contacting the third spatial-proximal digested ends of cross-linked DNA molecules with ligase, thereby generating third cross-linked proximity-ligated DNA molecules comprising first, second and third ligation junctions; (g) contacting the third cross-linked proximity-ligated DNA molecules comprising first, second and third ligation junctions with a fourth restriction endonuclease, thereby generating fourth spatial-proximal digested ends of cross-linked DNA molecules; (h) contacting the fourth spatial-proximal digested ends of cross-linked DNA molecules with ligase, thereby generating fourth cross-linked proximity-ligated DNA molecules comprising first, second, third and fourth ligation junctions; (i) contacting the fourth cross-linked proximity-ligated DNA molecules comprising first, second, third and fourth ligation junctions with a reagent that reverses cross-linking, thereby generating proximity-ligated DNA molecules comprising first, second, third and fourth ligation junctions; and (j) fragmenting the proximity-ligated DNA molecules to generate fragments of proximity-ligated DNA molecules comprising fragments spanning the first, second, third and fourth ligation junctions, wherein fragments spanning the first, second, third and fourth ligation junctions and of lengths that can be templates for short range sequencing, comprise sequences of essentially the whole genome or portion thereof

Also provided in certain aspects is a method for preparing DNA molecules from a sample comprising: (a) contacting cross-linked DNA molecules of a sample comprising a genome or portion thereof with a set of four restriction endonucleases; thereby generating spatial-proximal digested ends of cross-linked DNA molecules; (b) contacting the spatial-proximal digested ends of cross-linked DNA molecules with one or more reagents that incorporate biotin-attached to a nucleotide into the spatially-proximal digested ends, thereby generating cross-linked DNA molecules comprising labelled spatially-proximal digested ends; (c) contacting the cross-linked DNA molecules comprising labelled spatially-proximal digested ends with ligase, thereby generating cross-linked proximity-ligated DNA molecules comprising labelled ligation junctions; (d) contacting cross-linked proximity-ligated DNA molecules comprising labelled ligation junctions with a reagent that reverses cross-linking, thereby generating proximity-ligated DNA molecules comprising labelled ligation junctions; (e) fragmenting the proximity-ligated DNA molecules comprising labelled ligation junctions to generate fragments of proximity-ligated DNA molecules comprising fragments spanning the labelled ligation junctions, wherein fragments spanning the ligation junctions and of lengths that can be templates for short range sequencing, comprise sequences of essentially the whole genome or portion thereof; and (f) enriching for DNA fragments spanning the labelled ligation junctions by affinity purification of labelled ligation junctions using an affinity purification molecule comprising streptavidin.

Also provided in certain aspects is a method for preparing DNA molecules from a sample comprising (a) contacting spatially-proximal DNA molecules with stable spatial interactions from a sample, with two or more restriction endonucleases, thereby digesting the DNA molecules and generating spatial-proximal digested ends of DNA molecules; and (b) contacting the spatial-proximal digested ends of DNA molecules with ligase, thereby generating proximity-ligated DNA molecules comprising ligation junctions, wherein the ligation junctions are unmarked.

Also provided in certain aspects is a method for preparing DNA molecules from a sample comprising (a) contacting spatially-proximal DNA molecules with stable spatial interactions that are within cells/nuclei from a sample, with two or more restriction endonucleases, thereby digesting the DNA molecules and generating spatial-proximal digested ends of DNA molecules; and (b) contacting the spatial-proximal digested ends of DNA molecules with ligase, thereby generating proximity-ligated DNA molecules comprising ligation junctions, wherein the ligation junctions are unmarked and the contacting steps are in situ.

Also provided in certain aspects is a method for preparing DNA molecules from a sample comprising (a) contacting spatially-proximal DNA molecules with stable spatial interactions from a sample, with a first restriction endonucleases, thereby digesting the DNA molecules and generating first spatial-proximal digested ends of DNA molecules; (b) contacting the first spatial-proximal digested ends of DNA molecules with ligase, thereby generating first proximity-ligated DNA molecules comprising first ligation junctions, wherein the ligation junctions are unmarked; (c) contacting the first proximity-ligated DNA molecules comprising first ligation junctions with a second restriction endonuclease, thereby digesting the first proximity-ligated DNA molecules and generating second spatial-proximal digested ends of DNA molecules and (d) contacting the second spatial-proximal digested ends of DNA molecules with ligase, thereby generating second proximity-ligated DNA molecules comprising first and second ligation junctions, wherein the ligation junctions are unmarked.

Also provided in certain aspects is a method wherein (e) the second proximity-ligated DNA molecules comprising first and second ligation junctions are contacted with a third restriction endonuclease, thereby digesting the second proximity-ligated DNA molecules and generating third spatial-proximal digested ends of DNA molecules and (f) contacting the third spatial-proximal digested ends of DNA molecules with ligase, thereby generating third proximity-ligated DNA molecules comprising first, second and third ligation junctions, wherein the ligation junctions are unmarked.

Also provided in certain aspects is a method for preparing DNA molecules from a sample comprising (a) contacting spatially-proximal DNA molecules with stable spatial interactions that are within cells/nuclei from a sample, with a first restriction endonucleases, thereby digesting the DNA molecules and generating first spatial-proximal digested ends of DNA molecules; (b) contacting the first spatial-proximal digested ends of DNA molecules with ligase, thereby generating first proximity-ligated DNA molecules comprising first ligation junctions, wherein the ligation junctions are unmarked and the contacting steps are in situ; (c) contacting the first proximity-ligated DNA molecules comprising first ligation junctions with a second restriction endonuclease, thereby digesting the first proximity-ligated DNA molecules and generating second spatial-proximal digested ends of DNA molecules and (d) contacting the second spatial-proximal digested ends of DNA molecules with ligase, thereby generating second proximity-ligated DNA molecules comprising first and second ligation junctions, wherein the ligation junctions are unmarked and the contacting steps are in situ.

Also provided in certain aspects is a method wherein (e) the second proximity-ligated DNA molecules comprising first and second ligation junctions are contacted with a third restriction endonuclease, thereby digesting the second proximity-ligated DNA molecules and generating third spatial-proximal digested ends of DNA molecules and (f) contacting the third spatial-proximal digested ends of DNA molecules with ligase, thereby generating third proximity-ligated DNA molecules comprising first, second and third ligation junctions, wherein the ligation junctions are unmarked and the contacting steps are in situ.

Also provided in certain aspects are methods utilizing the above-described optimized 3C protocols with applications that benefit from increased coverage uniformity of read-pairs containing ligation junctions such as clustering, ordering, and orienting contigs in a genome, metagenome assemblies and haplotype phasing.

Also provided in certain aspects are methods utilizing the above-described optimized 3C protocols with applications that depend on 1D genome coverage uniformity such as SNV discovery, breakpoint detection, base polishing genome assemblies, and 1D “peak calling”, such as in ChIP-seq.

Also provided in certain aspects are methods utilizing the above-described optimized 3C protocols with applications that benefit from increased ligation events that preserve spatial-proximal contiguity information such as detection of pairwise 3D genome interactions and 3D conformation analysis.

Also provided in certain aspects are libraries prepared utilizing the methods described herein.

Also provided in certain aspects are kits comprising reagents for performing the methods described herein.

Also provided in certain aspects are methods of obtaining spatial positioning of sequence information obtained from a proximity-ligated tissue section 3C or HiC).

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows capturing spatial-proximal contiguity information via PL (Proximity Ligation) methods.

FIGS. 2A and 2B show ultra-high RE cut site density enables uniform genome coverage.

FIGS. 3A and 3B show the selection of optimal restriction enzymes.

FIG. 4 shows equivalent SNV discovery performance compared to shotgun WGS in four individuals.

FIGS. 5A and 5B illustrate more precise genomic rearrangement breakpoint detection

FIGS. 6A to 6D illustrate more comprehensive contig clustering and more accurate contig ordering.

FIG. 7 illustrates more accurate contig orientations.

FIGS. 8A and 8B illustrate higher resolution 3D genome conformation analysis.

FIG. 9 illustrates highly sensitive protein factor location and 3D conformation analysis.

FIG. 10 illustrates highly sensitive and concurrent variant discovery and haplotype phasing analysis.

FIG. 11 illustrates improved preservation of spatial-proximal contiguity in nucleic acid templates via multi-enzyme 3C implemented as simultaneous digestions.

FIG. 12 illustrates improved preservation of spatial-proximal contiguity in nucleic acid templates via multi-enzyme 3C implemented as sequential digestions.

FIG. 13 illustrates improved preservation of spatial-proximal contiguity in nucleic acid templates via size selection of large fragments in a 3C library.

FIGS. 14A and 14B illustrate that HiCoverage enables nearly complete genomic coverage across a range of plant and animal species. FIG. 14A is directed to vertebrate genomes. FIG. 14B is directed to insect, plant and parasite genomes.

FIG. 15 illustrates that HiCoverage enables uniform genomic coverage.

FIGS. 16A and 16B illustrate improved preservation of spatial-proximal contiguity and genomic coverage of ligation-junction containing nucleic acid templates via multi-enzyme 3C implemented as sequential rounds of digestion and ligation. FIG. 16A illustrates the size of digested and ligated products. FIG. 16B illustrates the % long-range cis read-outs.

### DETAILED DESCRIPTION

Provided herein are methods and compositions for preparing sequencing templates that provide uniform genome coverage and preserve spatial-proximal contiguity information.

Proximity Ligation

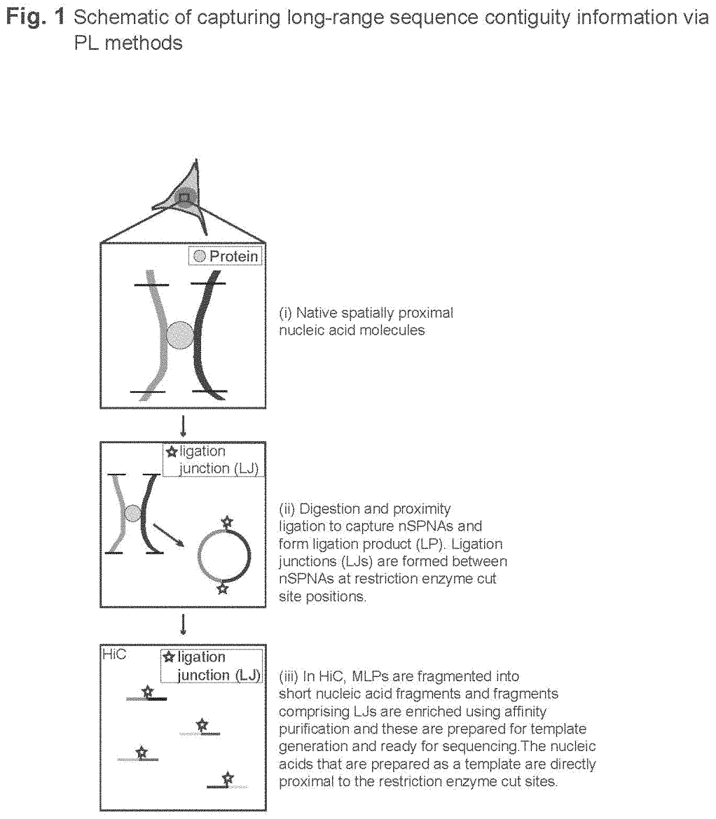

PL methods (see FIG. 1) begin with (i) native spatially-proximal nucleic acids (nSPNAs) within a nucleic acids source (e.g. nuclei, cells, tissues, FFPE samples), which are cross-linked followed by (ii) digestion (e.g. via RE, see black tick marks) of chromatin of the solubilized and decompacted sample and ligation of spatially-proximal digested end to generate ligation products (LPs), whereby the ligation junction manifests at the respective RE cut site locations from each ligated nSPNA and preserves spatial-proximal contiguity information. Broadly, PL methods are classified as 3C-based and HiC-based, although there are many specific variations of PL.

In 3C, the plurality of LPs are fragmented, prepared as short nucleic acid templates and ready for sequencing. In 3C, the nucleic acid template comprises nucleic acids that are proximal to RE cut sites, and distal to RE cut sites. (Dekker et al. Science 295, 1306-1311 (2002))

In HiC, the digested nucleic acid ends are marked (e.g. biotinylated) and then ligated to create marked ligated products (MLPs, MLPs are a manifestation of LPs), bearing an affinity purification marker at the ligation junctions (LJs). After the plurality of MLPs are fragmented, affinity purification is used to enrich for fragments of MLPs comprising Us and such fragments are prepared as nucleic acid templates and are ready for sequencing—i.e. the fragmented nucleic acids from the MLPs that contain at least an LJ are enriched and prepared as a template and sequenced in HiC, to deplete uMLPs (unligated MLPs that do not usually manifest LJs). Because of this enrichment for Us, the nucleic acid template only comprises nucleic acids that are proximal to RE cut sites. (see Lieberman-Aiden et al. US2017/0362649, Lieberman-Aiden et al. Science 326, 289-293 (2009), Dekker et al. (U.S. Pat. No. 9,434,985)).

In some embodiments, of a proximity ligation method, often includes steps: (1) digestion of chromatin of the solubilized and decompacted sample with a restriction enzyme (or fragmentation); (2) blunting the digested or fragmented ends or omission of the blunting procedure; and (3) ligating the spatially-proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library.

In some embodiments, the proximity-ligated nucleic acid molecules are generated in situ. As used herein the term “in situ” refers to within a nucleus (see U.S. Application US2017/0362649).

In some embodiments, proximity-ligated DNA molecules are analyzed in a chromatin conformation assays other than 3C or HiC. In some embodiments, the chromatin conformation assay is Capture-C(Hughes et al. Nature genetics, 46(2), p. 205 (2014) 4C (Simonis et al. Nature Genetics 38, 1348-1354 (2006), De Laat et al. (U.S. Pat. No. 8,642,295)), 5C (Dostie et al. Genome Research 16, 1299-1309 (2006), Dekker et al. (U.S. Pat. No. 9,273,309)), Capture-HiC (Jäger et al. Nature communications, 6, p. 6178 (2015)), HiChIP (Mumbach et al. Nature methods, 13(11), pp. 919-922 (2016)), PLAC-seq (Fang et al. Cell research, 26(12), pp. 1345-1348 (2016)), tethered chromosome capture (TCC) (Kalhor et al. Nature Biotechnology 30, 90-98 (2012), Chen et al. (US20110287947)), HiCulfite (Stamenova et al. bioRxiv, p. 481283 (2018)) Methyl-HiC (Li et al. Nature methods, 16(10), pp. 991-993 (2019)), HiChIRP (Mumbach et al. Nature methods, 16(6), pp. 489-492 (2019)) or combinations thereof.

Regardless of the specific PL method, all PL methods capture spatial-proximal contiguity information in the form of ligation products, whereby a ligation junction is formed between two natively spatially-proximal nucleic acids. Once the LPs are formed, the spatial-proximal contiguity information is detected using next generation sequencing, whereby one or more ligation junctions (either from an entire LP or fragment of an LP) are sequenced (as described herein). With these sequence information, one is informed that the nucleic acid molecules from a given ligation product (or ligation junction) are natively spatially-proximal nucleic acids.

In certain embodiments, wherein the assay is genome-wide (i.e., is directed to the whole genome). In some embodiments, the assay is 3C, HiC, tethered chromosome capture (TCC), HiCulfite, Methy-HiC or combinations thereof.

In certain embodiments, the assay is directed to one or more target regions in the genome. In some embodiments, the assay is Capture-C, 4C, 5C, Capture-HiC, HiChIP, PLAC-seq, HiChIRP or combinations thereof. In some embodiments the targets are single nucleotide variations, insertions, deletions, copy number variations, genomic rearrangements or targets for phasing. In some embodiments, the sample comprises a cancer genome and the target region is associated with a phenotype of the cancer. In some embodiments the target associated with the cancer is a structural variation such as a genomic rearrangement or a copy number variation. In certain embodiments the target is an oncogene or a panel of oncogenes.

Ultra-High Cut Site Density

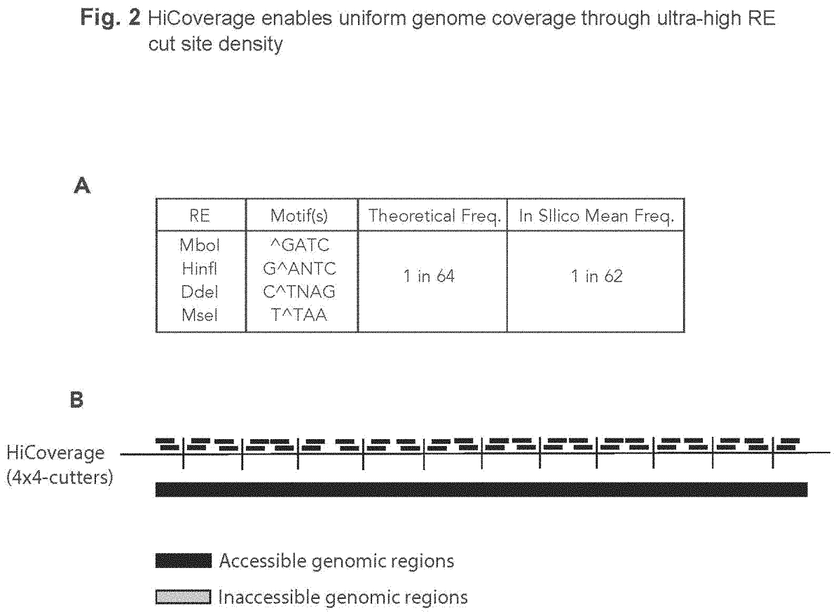

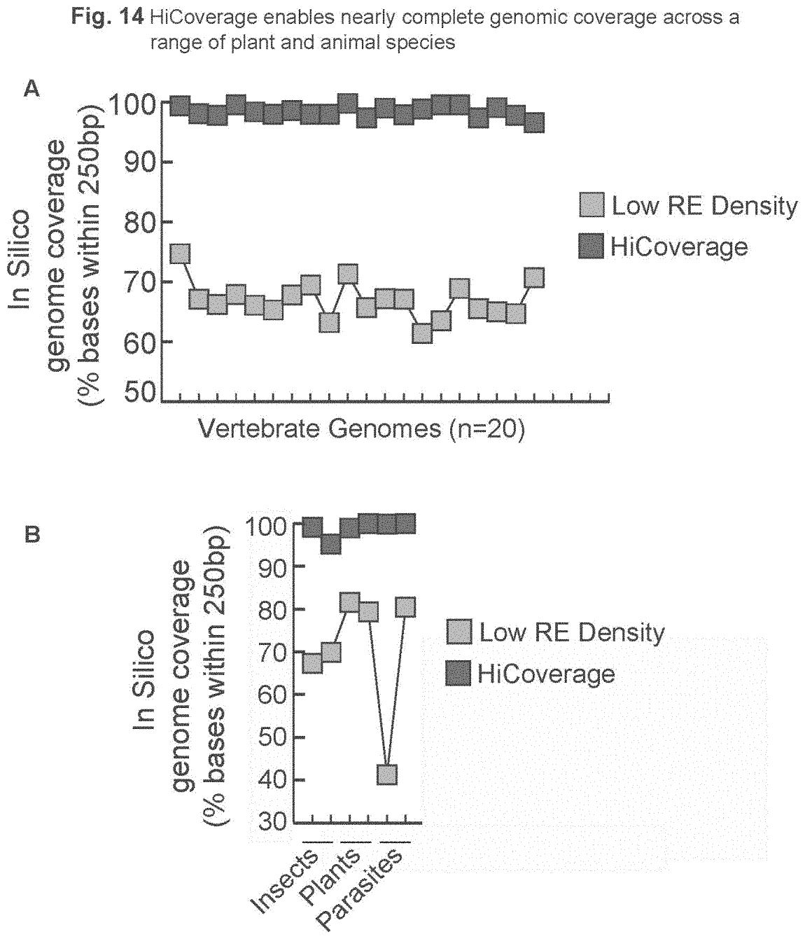

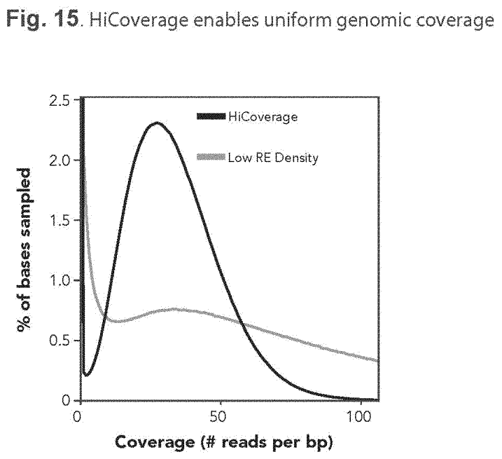

FIGS. 2A and 2B shows maximizing genome coverage by maximizing the amount of nucleic acids that are proximal to RE cut sites (ultra-high cut site density, “HiCoverage”) and thus would be represented in the HiC nucleic acid sequencing template. FIG. 2A is a table showing the RE motifs, theoretical RE digestion frequency, and in silico mean digestion frequency based on the human genome (hg19). In the methods described herein a cocktail of multiple RE 4-cutters is used to simultaneously digest the genome during HiC. This increases the RE cut site density by a log-order over that of standard HiC protocols, and in doing so maximizes the genome coverage and uniformity to a level comparable to shotgun WGS (see FIG. 2B) and enables data applications that benefit from or require uniform genome wide coverage (also see Example 3 and FIGS. 14A and 14B, complete genome coverage across a range of plant and animal species coverage and Example 4 and FIG. 15, coverage uniformity). The maximized genome coverage and uniformity is represented in the fragments of proximity-ligated DNA molecules spanning ligation junctions. The distribution of the ligation junctions in the genome is the result of the ultra-high cut site density of the described method. The fragments of proximity-ligated DNA molecules spanning the ligation junctions comprise sequences of essentially the whole genome or a portion thereof. In some embodiments, fragments spanning the ligation junctions and of lengths that can be templates for short range sequencing, comprise sequences of essentially the whole genome or portion thereof. In certain embodiments, the fragments spanning the ligation junctions comprise fragments up to 750 base pairs.

Restriction Endonucleases

In some embodiments, restriction endonucleases used in the described methods each have a theoretical digestion frequency of about 1 in 256 and when four are combined have a theoretical digestion frequency of about 1 in 64. However, there is a discrepancy between the theoretical digestion frequency, the predicted in silico frequency and the observed fragment size after chromatin digestion. Theoretical digestion frequency and in silico frequency are poor predictors of how a given restriction endonuclease will digest chromatin and particularly cross-linked chromatin.

In some embodiments, cross-linked DNA molecules of a sample are contacted with a set of restriction endonucleases so that each restriction endonuclease functions to digest the cross-linked DNA molecules during approximately the same period of time. In some embodiments, restriction endonucleases of a set each have a high activity level (i.e., approximately 100% of optimum cutting efficiency) in a common buffer. An examples of a common buffer is CutSmart™ (New England Biolabs, Beverly, Mass.).

In some embodiments restriction endonucleases can result in DNA molecules with 5′ overhangs, 3′ overhangs or no overhang (i.e., blunt ends).

In some embodiments, a set of restriction endonuclease can be at least three restriction endonucleases. In certain embodiments, a set of restriction endonucleases consists of four restriction endonucleases. In some embodiments, a sample comprises a genome other than a bacterial genome and a set of restriction endonucleases are selected to digest that genome. In certain embodiments, the four restriction endonucleases are: MboI, HinfI, MseI and DdeI. In some embodiments, a sample comprises one or more bacterial genomes, as in a metagenomics sample, and a set of restriction endonucleases are selected to digest the one or more bacterial genomes. In certain embodiments, the four restriction endonucleases are: HpyCH4IV, HinfI, HinP1I and MseI.

In some embodiments, the restriction endonucleases can be added to a sample sequentially and do not digest the cross-linked DNA molecules in the sample at the same time. In some embodiments, the restriction endonucleases generate DNA molecules with the same type of ends. In some embodiments, two or more of the restriction endonucleases generate DNA molecules with different types of ends (e.g., 5′ overhang, 3′ overhang, no overhang or blunt). In some embodiments, one or more of the restriction endonucleases require a specific buffer for high activity level that is different from a buffer required for high activity level of another of the restriction endonucleases. As the restriction endonucleases are individually contacting the cross-linked DNA molecules in the sample, each restriction endonuclease can be provided with its own unique buffer, if required. In certain embodiments, restriction endonucleases that are sequentially added to a sample can generate digested ends that can incorporate a different labelled nucleotide from a labelled nucleotide incorporated in a digested end generated by a different restriction endonuclease. This is in distinction with the use of restriction endonucleases that simultaneously digest the DNA molecules of a sample, which are limited to incorporating a common labelled nucleotide in the various digested ends.

Sequencing

Nucleic acid template (or “template” for short) refers to the nucleic acid molecule(s) that are read by a sequencing instrument. The process of generating nucleic acid templates often involves nucleic acid fragmentation to a molecular length recommended for a specific sequencing instrument. For example, current Illumina short-read sequencing can accommodate nucleic acid lengths (sequence template molecules) up to approximately 750 bp. Although smaller sequence template molecules can be utilized, as increasing the sequence coverage further away form cut sites should maximize genome coverage, templates molecules up to approximately 750 bp are often used. Templates comprise fragments that span ligation junctions and sequence information on both sides of a ligation junction can be obtained. However, as DNA shearing or fragmentation is random, the ligation junction can occur at any point along the template molecule. In some cases it may be very much towards the end of the molecule, such that there are only ˜20 bp on one side of the junction, and hundreds of bp on the other side of the junction. The junction can also occur in the middle of the template, such that there are a couple/few hundred base pairs on each side of the ligation junction.

Reads lengths can be can any length including but not limited to 2×150 bp, 2×100 bp, 2×75 bp or 2×50 bp.

In some embodiments, in order to maximize the quantity of sequence information obtained that spans a ligation junction the fragmented proximity-ligated molecules are enriched for fragmented proximity-ligated DNA molecules comprising ligation junctions and the fragmented proximity-ligated DNA molecules comprising ligation junctions are used to prepare a library of template molecules for DNA sequencing. In certain embodiments, the ligation junctions are marked with an affinity purification marker. In some embodiments, the affinity purification marker is biotin conjugated to a nucleotide. In some embodiments, spatial-proximal digested ends having a 5′ overhang are filled in by a polymerase such as Klenow Large Fragment using a single labeled-nucleotide (biotin labeled nucleotide) and other unlabeled nucleotides. In some embodiments, spatial-proximal digested ends having a 3′ overhang can be end labelled using an enzyme such as T4 DNA polymerase and all four nucleotides that are biotin labeled. In certain embodiments, enrichment is by affinity purification of the affinity purification marker with an affinity purification molecule. In some embodiments, affinity purification of the affinity purification marker with an affinity purification molecule is used in HiC, Capture-HiC, HiChIP, PLAC-seq, HiCulfite or Methyl-HiC. In some embodiments, the affinity purification molecule is streptavidin. In certain embodiments, the streptavidin comprises streptavidin coated on a magnetic bead.

In certain embodiments, enrichment for fragmented proximity-ligated DNA molecules comprising ligation junctions does not utilize a label incorporated into the ligation junction. In some embodiments, ends of molecules having 5′ or 3′ overhangs could be blunted without labeling and enriched by size selection. After the ligation step any DNA molecule that represents a proximity-ligated molecule with a ligation junction will be larger than a fragment that is unligated but digested. In some embodiments, the enriched by size selection proximity-ligated DNA molecules comprising ligation junctions are used in 3C-seq, 4C-seq 5C or Capture-C.

In some embodiments, the library of template molecules provides uniform genome-wide coverage of a genome or portion thereof. In some embodiments, the library of template molecules is sequenced to generate sequence reads comprising sequence information. In certain embodiments, the sequencing is short read sequencing.

In some embodiments, the sequence information is used in analysis of a genome. In some embodiments, the sequence information is used in analysis of a portion of a genome, for example in a targeted assay. In both analysis of a genome and analysis of a portion of a genome the uniformity and extent of coverage is the same

In some embodiments, the sequence information is utilized in genomic rearrangement analysis, identification of a breakpoint, clustering and ordering of contigs, determining contig orientation, clustering, ordering and orienting contigs, detection of pairwise 3D genome interactions (such as 3D genome interaction is between promoters, enhancers, gene regulatory elements, GWAS loci, chromatin loop and topological domain anchors, repetitive elements, polycomb regions, gene bodies, exons or integrated viral sequences), protein factor location analysis and 3D conformation, protein factor location analysis and 3D conformation analysis comprising PLAC-seq or HiChIP, haplotype phasing, genome assembly and 3D conformation analysis, DNA methylation analysis, DNA methylation analysis and detection of 3D genome interactions, single nucleotide variant (SNV) discovery, base polishing of long-range sequencing information, highly sensitive copy number variation (CNV) analysis (e.g., the copy number variation (CNV) is an amplification, the copy number variation (CNV) is a heterozygous or homozygous deletion), variant discovery, haplotype phasing and genome assembly, haplotype phasing and genome assembly, genome assembly and detection of 3D genome interaction or combinations thereof.

In certain embodiments, the sequence information is utilized for variant discovery and haplotype phasing in a first sample comprising a paternal genome and a second sample comprising a maternal genome and the phased variants of the paternal genome and the maternal genome are used to analyze sequence data of a fetal genome obtained from cfDNA of the mother.

Full genome coverage and spatial-proximal contiguity information obtained by the methods described herein can be used in other methods or combinations of methods that utilize such sequence information.

Samples

In some embodiments, the DNA is obtained from a sample selected from nuclei, cells, tissues, formalin-fixed paraffin-embedded (FFPE) samples, deeply formalin-fixed samples or cell-free DNA. In certain embodiments, the DNA is obtained from a single cell. In certain embodiments, the DNA is obtained from two or more cells. In some embodiments, a sample can comprise two or more genomes representing different species, such as in a metagenomics sample.

Genomic Rearrangement Breakpoint Detection

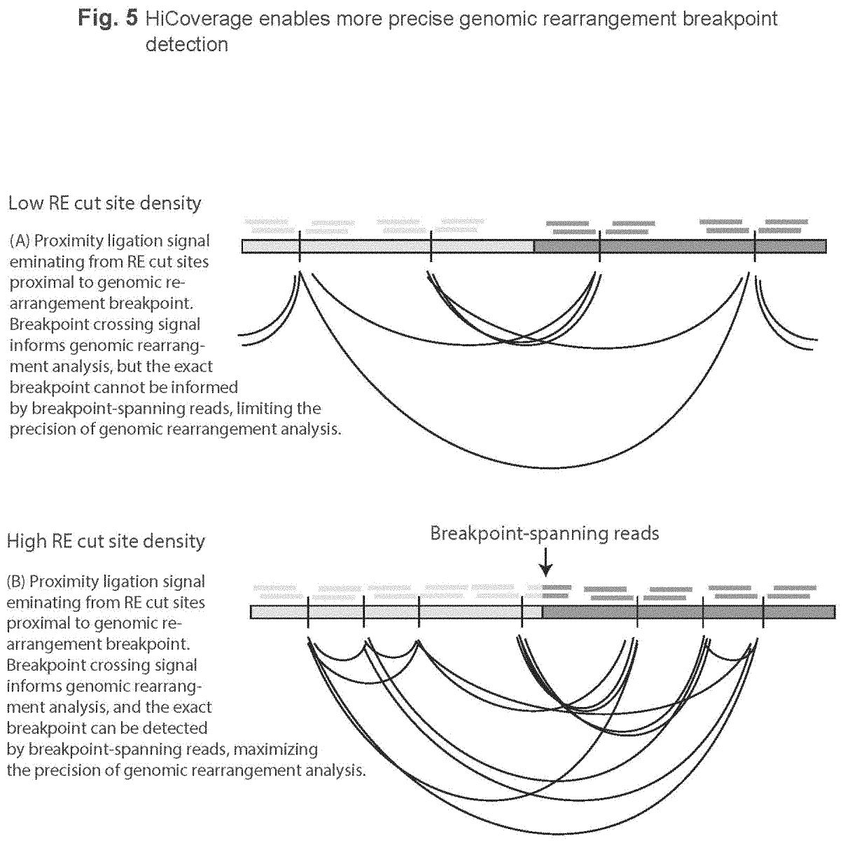

FIGS. 5A and 5B show how ultra-high RE cut site density (HiCoverage) enables more precise genomic rearrangement analysis compared to previous HiC methods. In FIG. 5A, when the RE cut site density is low, such as in previous HiC methods (Lieberman-Aiden, Science, 2009; Rao, Cell, (2014)), the long-range “links” manifested in the ligation junction-containing nucleic acid templates (see arcs) inform the approximate location of a genomic rearrangement breakpoint by capturing signal that “crosses over” the genomic breakpoint. While this helps define the approximate location of the breakpoint, there are no nucleic acid template molecules that span across the breakpoint because the breakpoint lies distal to a RE cut site, and therefore the precision of such analysis is limited by sequence coverage. In FIG. 5B, ultra-high RE cut site density (HiCoverage) also comprises long-range “links” manifested in the ligation junction-containing nucleic acid templates (see arcs) that inform the approximate location of a genomic rearrangement breakpoint by capturing signal that “crosses over” the genomic breakpoint, but also the increased RE cut site density allows for chimeric nucleic acid template molecules that span the genomic rearrangement breakpoint to enable breakpoint precision analyses.

Contig Clustering and Ordering

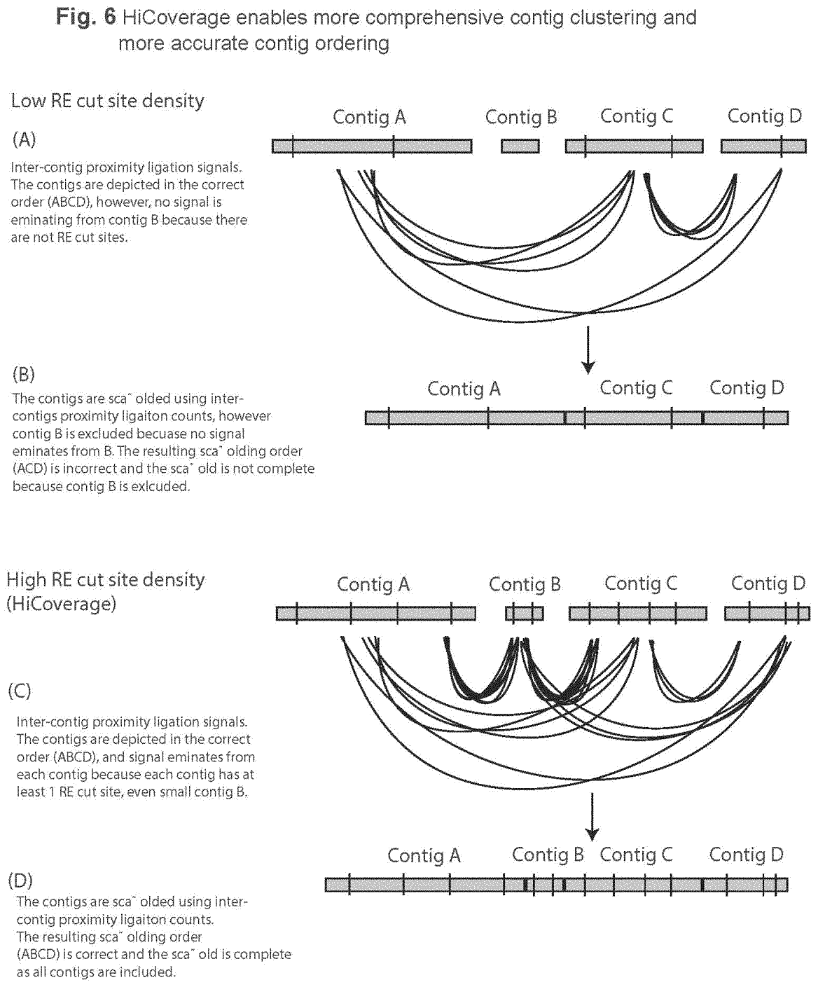

FIGS. 6A-6D show how maximizing genome coverage in ultra-high RE cut site density (HiCoverage) uniquely enables more inclusive (i.e. more complete) clustering of contigs into chromosomes, and thus more accurate contig ordering in the genomic (or metagenomic) assembly. In one scenario, de novo genome assembly workflows often involve the combination of long-read sequencing technology (e.g. Oxford Nanopore, UK) to produce the most contiguous sequences (“contigs”), followed by performing HiC. The first function of the HiC data (sequence information from ligation junctions) is to use the inter-contig long-range “links” manifested in the ligation junction-containing nucleic acid templates (see arcs) to inform which contigs are derived from the same chromosome in the genome assembly case, or the same organism in the metagenome assembly case. The HiC data therefore is said to “cluster” the contigs. Once clustered, the frequency of the pairwise long-range “links” between contigs is used to determine the relative ordering of the contigs along the chromosome based on the premise that frequently occurring spatially-proximal contigs captured by HiC should also be linearly proximal due to the properties of polymer physics. In FIG. 6A, the low RE cut site density produced by existing HiC methods can lead to certain contigs being devoid of a RE cut site, and then not represented in the nucleic acid template or sequencing data. When the long-range “links” between contigs are used to cluster contigs into chromosome(s), contigs without a RE cut site cannot be clustered, thus leading to incomprehensive or incomplete chromosomal sequence content. As a byproduct of this incomplete clustering, the ordering of contigs will also be incorrect. In FIG. 6A, Contigs C and D, and A and C has the most frequent inter-contig links, while A and D have the fewest. Using this information, in FIG. 6B, the order of such contigs may be inferred as ACD, with B excluded and thus producing an erroneous contig order. In FIG. 6C, coverage uniformity via ultra-high RE cut site density (HiCoverage) enables the capture of long-range inter-contig links between all contigs, enabling the comprehensive and complete clustering of contigs to chromosomes. In FIG. 6D, because of the complete contig clustering, all contigs are available for analysis of contig order based on inter-contig link frequency, and the correct contig order can be derived (ABCD).

Contig Orientation

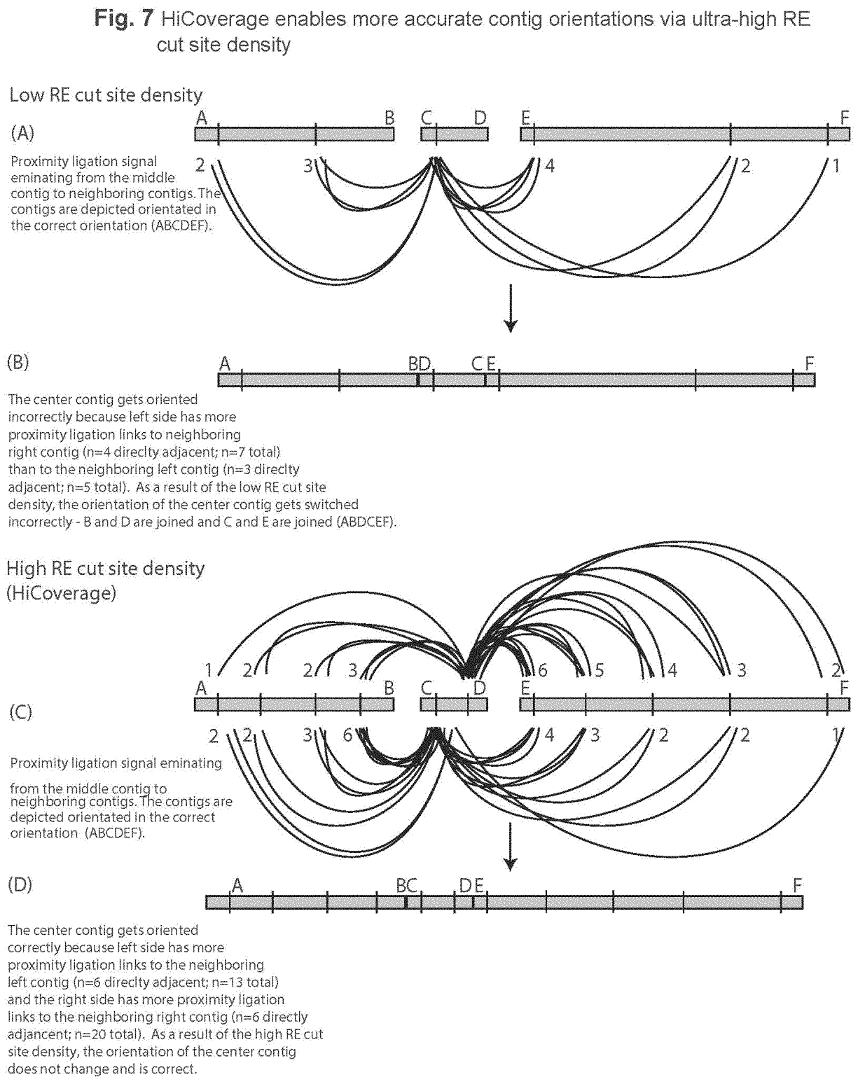

FIGS. 7A-7D show how maximizing genome coverage in ultra-high RE cut site density (HiCoverage) uniquely enables more accurate contig orientation analysis. In the de novo genome assembly scenario previously described (see FIGS. 6A to 6D), the next utility of HiC data after contig ordering analysis is contig orientation analysis to determine which ends of neighboring contigs should be joined. This can be determined by analyzing the frequency of links between the ends of neighboring contigs, and is also based on the premise that frequently occurring inter-contig links captured by HiC should also be linearly proximal due to the properties of polymer physics. In other words, the two neighboring contigs ends with the highest inter-contig link frequency should be orientated in such a way that those two ends are joined. To illustrate this concept, inter-contig HiC link information between a center contig and two neighboring contigs is shown in FIGS. 7A-7D. Each end of the contigs are labeled with a letter to assign an ID to each contig end and the correct order is depicted as ABCDEF with inter-contig HiC link frequency information (see arcs). In FIG. 7A, the infrequent and uneven RE cut site density results in inter-contig HiC links emanating from only the left end of the center contig. The inter-contig link frequency is greatest between C and E, not C and B, informing that contigs ends C and E should be erroneously joined, producing the incorrect contig orientation (ABDCEF) (see FIG. 7B). In FIG. 7C, coverage uniformity via ultra-high RE cut site density (HiCoverage) enables greater inter-contig HiC links emanating from the center contig, as well as adjacent contigs, such that link information from ends C and D can now inform contig orientation analysis. The top arcs depict the inter-contig HiC links emanating from D, and the lower arcs depict the inter-contig HiC links emanating from C. As depicted, the inter-contig link frequency between B and C, as well as D and E, are greatest (n=6, each), thus informing that those pairs of ends should be joined, producing the correct contig orientation (ABCDEF) (see FIG. 7D).

3D Genome Conformation

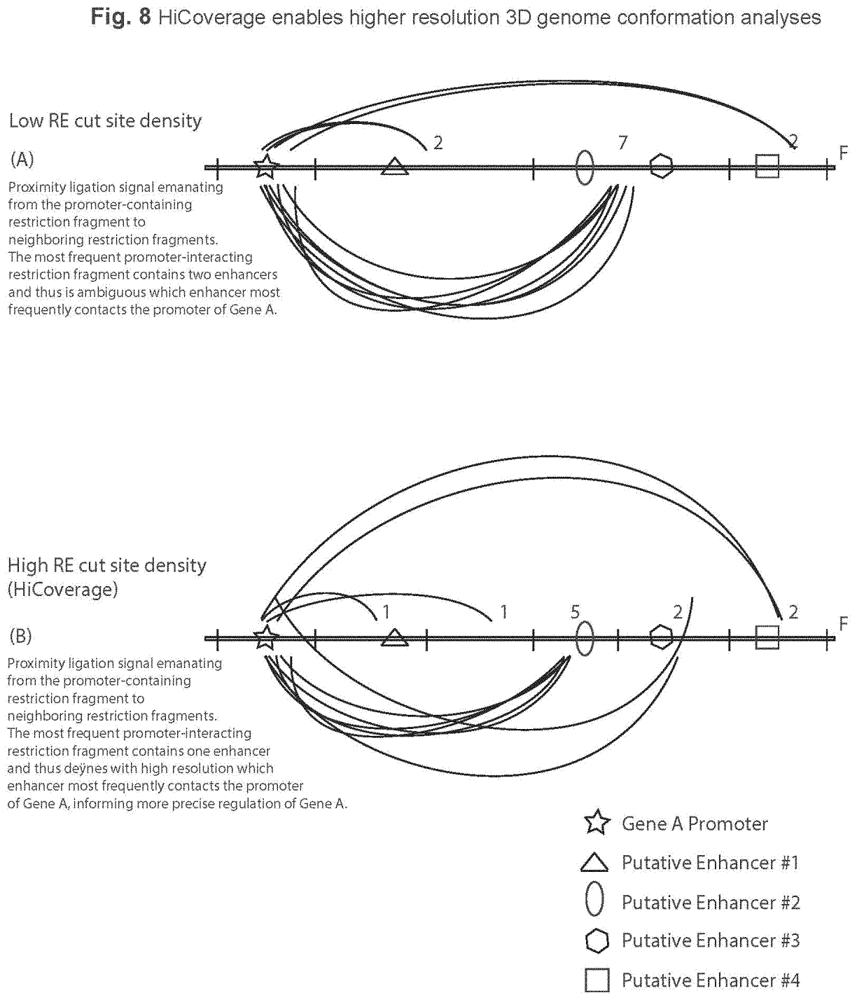

FIGS. 8A and 8B show how maximizing genome coverage in ultra-high RE cut site density (HiCoverage) uniquely enables highest resolution and most sensitive detection of pairwise 3D genome interactions. In 3D genome organization analysis using HiC, lower resolution HiC is often aggregated into fixed interval “bins” prior to analysis the bin-pair interaction frequency between any two bins. The highest resolution analysis afforded by HiC is “restriction fragment” level HiC analysis, whereby pairwise interaction frequency between individual restriction fragments is quantified and is therefore delimited by the frequency of the RE cut sites. However, RE with relatively low RE frequency can suffer from low resolution and imprecision when performing 3D genome analysis. In one scenario (see FIG. 8A), a promoter-containing restriction fragment appears to be frequently interacting with another downstream restriction fragment comprises two gene regulatory elements (putative enhancers). Because two enhancers are contained within the restriction fragment, it is unclear which enhancer would regulate Gene A. In FIG. 8B, the same total number of interactions emanate from the restriction fragment containing Gene A Promoter, however, they are now linked to more neighboring restriction fragments due to the higher RE cut site density. As depicted, the most frequent interaction is to the restriction fragment comprising putative enhancer #2, helping identify this as the target enhancer of Gene A, not the neighboring putative enhancer #3. Note that pairwise detection of promoter-enhancer interaction represents just one type of 3D interaction analysis. Other analyses include but are not limited to the pairwise interactions between promoters, enhancers, other gene regulatory elements, GWAS loci, chromatin loop and topological domain anchors, and other genomic elements or sequences of interest (e.g. repetitive elements, polycomb regions, gene bodies, exons, integrated viral sequences, etc.).

Maximizing genome coverage in ultra-high RE cut site density (HiCoverage) to uniquely enable the highest resolution and most sensitive detection of pairwise 3D genome interactions also applies to other forms of HiC and its derivatives, particularly Capture-HiC, HiChIP, TCC, and other restriction enzyme-based genome-wide or targeted HiC-based assays.

Protein Factor Localization

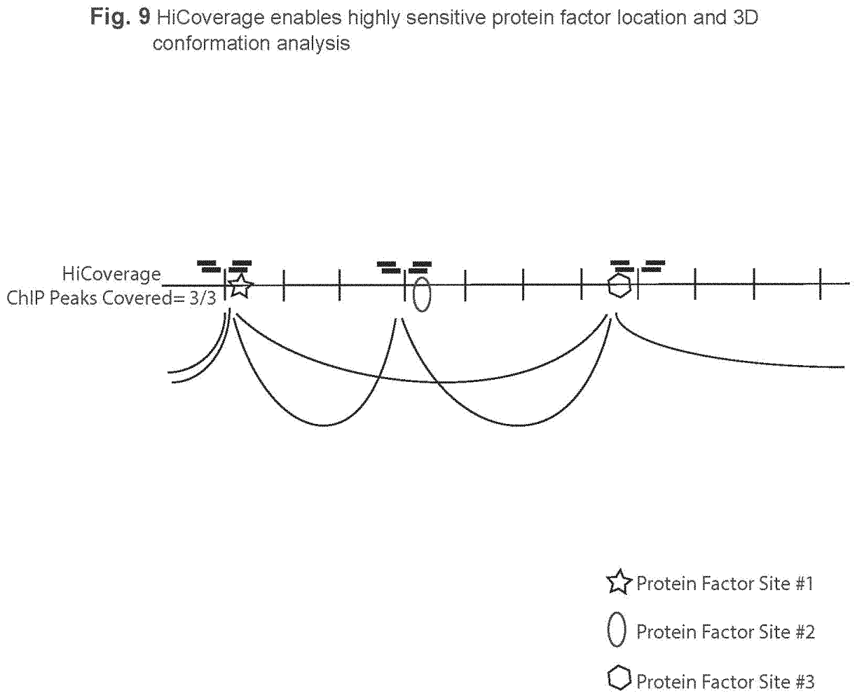

FIG. 9 shows how maximizing genome coverage in ultra-high RE cut site density (HiCoverage) uniquely enables more sensitive protein factor location analysis and 3D conformation analysis in HiChIP (PLAC-seq) assays. In the HiChIP assays, proximally-ligated chromatin is sheared and enriched for a protein factor of interest (CTCF, H3K27ac, cohesion subunit protein, H3K4me3, etc.). After purifying the protein bound ligated DNA, ligation junctions are enriched, producing a nucleic acid template that comprises ligation events mediated by a specific protein-factor. In doing so, HiChIP provides information not only on protein factor localization (similar to ChIP-seq), but also 3D genome conformation (similar to HiC). One main limiting factor is that in order for a nucleic acid to be prepared as a template, it must be linearly proximal to both a protein factor location site, and, a restriction enzyme cut site. In HiCoverage, increased RE cut site density results in a greater percentage of protein factor location sites represented in the nucleic acid template increases, and more unique ligation junctions emanating from protein factor localization sites. When these nucleic acid templates are sequenced, the sequence data derived from the nucleic acid templates facilitates more sensitive protein location analysis (e.g. 1D “peak calling”, such as in ChIP-seq) and more sensitive 3D interaction analysis (e.g. 2D “peak calling”, such as in HiC).

Variant Discovery and Haplotype Phasing

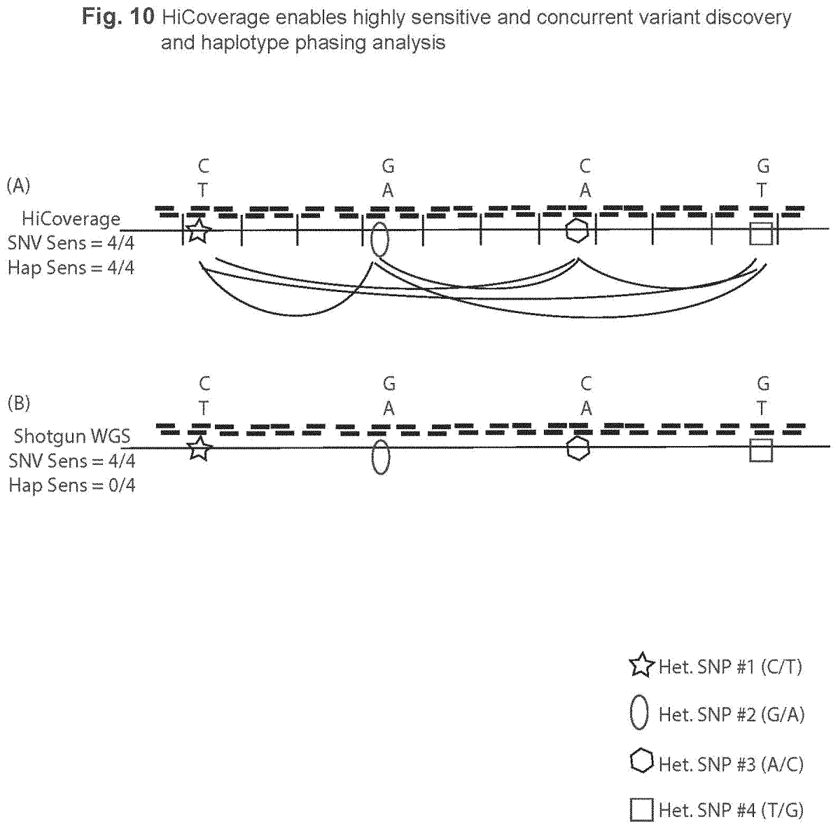

FIGS. 10A and 10B show how maximizing genome coverage in ultra-high RE cut density (HiCoverage) uniquely enables highly sensitive and concurrent variant discovery and haplotyping analysis. FIG. 10A shows the impact of this in the context of variant discovery and haplotype phasing, 4 Het. SNVs are depicted along a region of the genome. Het. SNVs obtains sequence coverage due to their close proximity to a RE cut site. If a Het. SNV is distal to a RE cut site, it receive no sequence coverage and therefore cannot be discovered. Also, only SNVs with long-range linkage information provided by HiC can be utilized for read-based haplotype phasing. HiCoverage coverage uniformity via ultra-high RE cut site density enables 4/4 Het. SNVs to obtain sequence coverage, thus maximizing small variant discovery sensitivity and haplotype phasing sensitivity. In FIG. 10B, shotgun WGS coverage uniformity is not confined to regions proximal to RE cut sites and thus also enables 4/4 Het. SNVs to obtain sequence coverage, thus maximizing small variant discovery sensitivity. However, shotgun WGS does not comprise long-range contiguity information, and thus 0/4 SNVs can be haplotyped. Note that heterozygous SNVs are depicted to illustrate of the variant sensitivity concept, but other types of variants can be concurrently discovered and haplotype phased with maximum sensitivity using ultra-high RE cut density (HiCoverage).

Methylation Analysis

Maximizing genome coverage in the HiCoverage method uniquely enables highly sensitive analysis of DNA methylation, and is comparable to traditional whole genome bisulfite sequencing (WGBS). Only cytosines that are proximal to a RE cut site would be present in a nucleic acid template and available for bisulfite conversion and determination of methylation status. Cytosines distal from a RE cut site would be unknown because those nucleic acids would not be present in the nucleic acid template. HiCoverage uniformity via ultra-high RE cut site density enables the methylation status of all cytosine to be detected due to their proximal positioning relative to RE cut sites. Other types of DNA methylation, e.g. hydroxymethylated cytosines, can also be sensitively detected using HiCoverage by virtue of the genome coverage (apply bisulfite conversion to one set of templates and apply TAB-seq to another set of templates and using the two datasets determine mC and hmC status).

In some embodiments the nucleic acids with preserved spatial-proximal contiguity information generated by the methods described herein are contacted with a bisulfite reagent prior to PCR and sequencing to enable the concurrent analysis of spatial proximity and DNA methylation at base resolution. In some embodiments the bisulfite reagent is sodium bisulfite.

In some embodiments HiC ligation products are generated using a HiC protocol as previously described (Rao et al. Cell, 159(7), pp. 1665-1680 (2014), Li et al. Nature methods, 16(10), pp. 991-993 (2018)). Ligation junctions are enriched using streptavidin beads. Illumina library construction ensues while the DNA is attached to the streptavidin bead, as previously described (Rao et al. Cell (2014)). Directly after adapter ligation, DNA is subject to bisulfite conversion, using methods known in the art. Unmethylated lambda DNA is spiked in at 0.5% prior to bisulfite conversion in order to estimate the conversion rate. The bisulfite converted DNA is purified, amplified, and sequenced.

In some embodiments sheared HiC ligation products are treated with a bisulfite reagent and purified (Stamenova et al. bioRxiv, p. 481283 (2018)). Ligation junctions are then enriched using streptavidin beads. DNA is then detached from the beads, and prepared as a sequencing library using techniques known in the art for converting ssDNA into a dsDNA sequencing library. Adapter ligated molecules are then subject to library amplification and sequencing. Similarly, methods known to the art can also be applied to analyze the DNA methylation status (Lister et al. Nature, 462(7271), pp. 315-322 (2009); Shultz et al. Nature, 523(7559), pp. 212-21 (2015)). Additionally, methods known in the art can also be applied to concurrently analyze the DNA methylation status with respect to 3D genome folding (Li et al. Nature methods, 16(10), pp. 991-993 (2018); Stamenova et al. bioRxiv (2018)), revealing DNA chemical modifications properties and DNA folding patterns in parallel. Specifically in the context of applying this method to protein:cfDNA complexes, it is well known in the art that DNA methylation status of cell free nucleic acids can inform tissue of origin analyses as well as several other cfDNA analysis, including but not limited to the non-invasive detection of tumor DNA, prenatal diagnoses, and organ transplantation monitoring (Zeng et al. Journal of Genetics and Genomics, 45(4), pp. 185-192 (2018); Lehmann-Werman et al. Proceedings of the National Academy of Sciences, 113(13), pp. E1826-E1834 (2016)).

SNV Discovery

Maximizing genome coverage (sequence coverage and uniformity) enables highly sensitive small variant sensitivity. A SNV obtains sequence coverage due to its close proximity to a RE cut site. A SNV that is distal to a RE cut site and receive no sequence coverage and therefore cannot be discovered. In the methods described herein, coverage uniformity via ultra-high RE cut site density enables essentially all SNVs to obtain sequence coverage, thus maximizing small variant sensitivity to an equivalent level as demonstrated with shotgun WGS. Standard HiC results in many SNV distal to an RE cut site, thus being undiscoverable. Many types of small variants, including heterozygous SNV (single nucleotide variations), other types of SNVs, and INDELs (insertions and deletions), can be discovered with maximum sensitivity using the described method.

Base Polishing

Maximizing genome coverage in the HiCoverage method uniquely enables highly sensitive base polishing of erroneous genomic bases, comparable to shotgun WGS, originally detected by error-prone sequencing technologies, in additional to the known genomic scaffolding capabilities of HiC. In one scenario, current de novo genome assembly workflows often involve the combination of a relatively error-prone long-read sequencing technology (e.g. Oxford Nanopore, UK) to produce the most contiguous sequences (“contigs”), followed by performing HiC to scaffold contigs into chromosome-scale scaffolds, followed by shotgun WGS (10× Genomics, Pleasanton, Calif.) to “polish” the erroneous base calls produced by the error-prone long-read technology. HiC has not been conceived as a technology capable of sensitive base polishing due to the uneven genomic representation in the nucleic acid template and thus the uneven coverage of the sequencing data. However using the HiCoverage method uniformity via ultra-high RE cut site density enables maximum base polishing sensitivity comparable to that of shotgun WGS. Oher types of erroneous DNA sequence, besides erroneous individual base calls, produced by error-prone sequencing technologies can also be sensitively polished using the HiCoverage method by virtue of the even genome coverage.

CNV Analysis

In some embodiments, Maximizing genome coverage in the HiCoverage method uniquely enables highly sensitive CNV analysis on bar with that of shotgun WGS. CNVs obtains sequence coverage due to its overlap with a RE cut site, while CNVs that are distal to a RE cut sites, receive no sequence coverage and therefore cannot be discovered or analyzed. The HiCoverage method provides coverage uniformity via ultra-high RE cut site density, thus maximizing CNV detection sensitivity. CNVs, such as amplified regions and heterozygous or homozygous deletions can be discovered and analyzed with maximum sensitivity using the described ultra-high RE cut site density method.

Data Analysis/Applications

The following represents a sampling of some data analysis and applications and is not meant to be all inclusive. Using HiC data for contiguity-preservation-enabled analysis and applications, such as haplotype phasing and genomic rearrangement detection is known to the art. For example, Selvaraj et al. BMC genomics, 16(1), p. 900 (2015), Selvaraj et al. Nature biotechnology, 31(12), p. 1111 (2013), and PCT/US2014/047243 described HiC data for haplotype phasing and Engreitz et al. (PLOS ONE September 2012/Volume 7/Issue 9/e44196) has described HiC data for genomic rearrangement analysis in human disease. Several other papers have described using HiC data for genomic rearrangement detection (Dixon et al. Nature genetics, 50(10), pp. 1388-1398 (2018); Chakraborty and Ay, Bioinformatics (2018); Harewood et al. Genome biology, 18(1), p. 125 (2017)). One such analysis tool for rearrangement detection is HiC-Breakfinder tool (https://github.com/dixonlab/hic_breakfinder) from Dixon et al. Nature genetics (2018). Other contiguity-preservation-enabled analyses and applications include but are not limited to de novo genome and metagenome assembly, structural variation detection, and others.

After sequencing, methods known to the art can be used to analyze the data in the context of spatial proximity and long-range sequence contiguity, such as but not limited to using the spatial-proximal contiguity information to inform genome folding patterns (Lieberman-Aiden et al. Science, 326(5950), pp. 289-293 (2009)), and genomic rearrangement analysis (Dixon et al, Nature genetics, (2018)).

Also, because it is known that HiC signal uniquely captures long-range sequence contiguity information to significantly enhance genomic rearrangement analyses (Dixon et al. Nature genetics (2018)), HiC applied to cfDNA could enrich for such genomic rearrangement signal from liquid biopsy samples and greatly benefit early non-invasive cancer diagnoses. And finally, the combination and concurrent analysis of both DNA methylation and DNA spatial proximity and long-range contiguity will synergize to better enable the analyses described herein.

3C Methods

In some embodiments, proximity ligation products are generated using optimized 3C-based methods, rather than a HiC method. 3C-based methods, include but are not limited to, 3C, 4C, 5C, Capture-C, 3C-ChIP or Methyl-3C.

In some embodiments, the 3C methods do not incorporate a label or marker in the ligation junction, as in HiC. For example, a biotinylated nucleotide or biotinylated bridge adaptor.

A sample is typically crosslinked to preserve spatial-proximal information, however crosslinking of a sample may not always be required (Bryant et al. Mol Syst Biol. 12(12): 891(2016)). In some embodiments, the 3C methods described herein are used with samples of tissues, cells, nuclei, that are not crosslinked, but which have spatially-proximal DNA molecules with stable spatial interactions. Embodiments of 3C methods described herein as applicable to crosslinked samples are also intended as applicable to samples that are not crosslinked.

The 3C methods described herein can be performed ex situ or in situ.

In some embodiments, 3C methods are optimized to improve amount of spatial-proximal contiguity information that is preserved. Long-range cis captured spatially-proximal nucleic acids (cSPNAs) (greater than 15 Kb in linear sequence distance) are most informative for contiguity applications and are often used as a proxy for determining the preservation of spatial-proximal contiguity information. Specifically what percent of nucleic acid templates for sequencing are long-range cis molecules. In certain embodiments, 3C methods are optimized to improve the percent of long-range cis molecules.

In some embodiments, the optimized 3C methods also increase genome coverage uniformity of read-pairs containing ligation junctions.

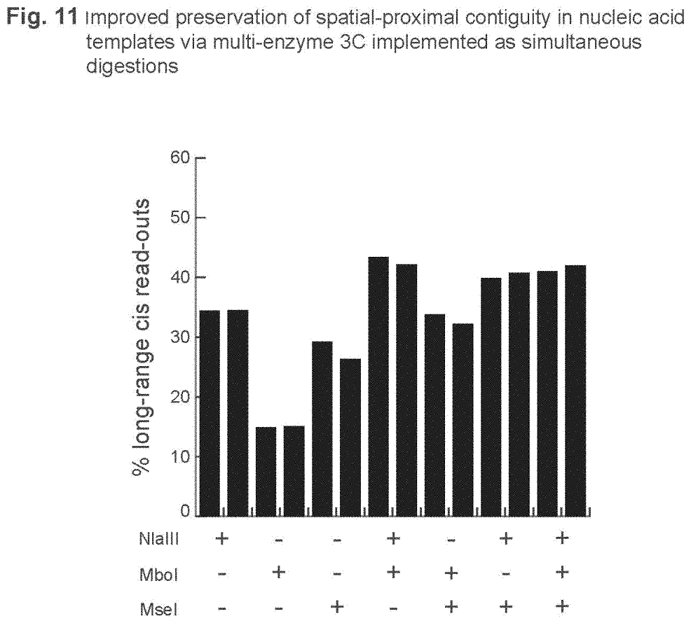

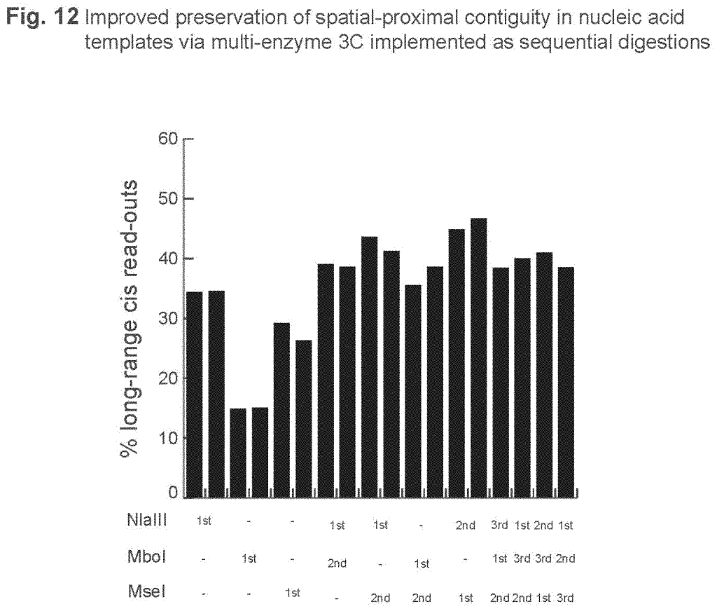

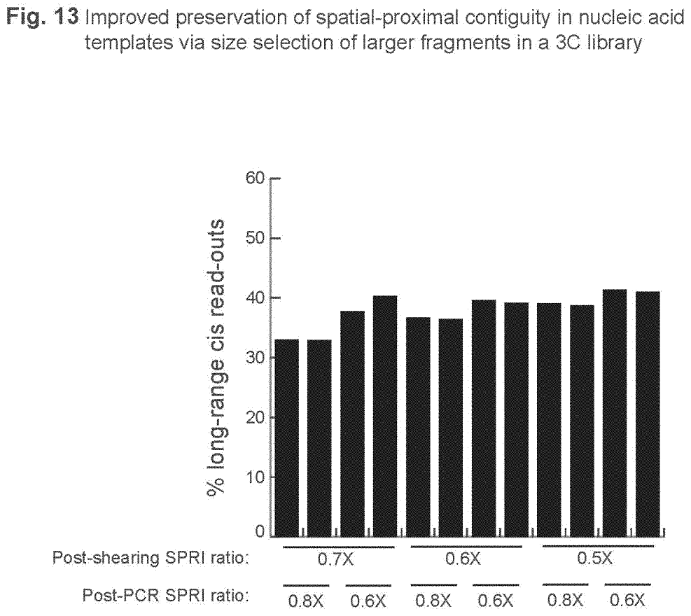

In some embodiments, optimized 3C is based on the use of multiple restriction endonucleases (optimized 3C proximity ligation) (see Examples 4 and 5 and FIGS. 11 and 12). In some embodiments, optimized 3C includes size selection for proximity-ligated molecules (see Example 7 and FIG. 13) along with the use of multiple restriction endonucleases.

Restriction Endonucleases

In some embodiments, DNA molecules of a sample are contacted and digested with two or more restriction endonucleases, three or more restriction endonucleases, four or more restriction endonucleases, five or more restriction endonucleases, six or more restriction endonucleases, seven or more restriction endonucleases, eight or more restriction endonucleases, nine or more restriction endonucleases, ten or more restriction endonucleases, or greater; e.g., 2, 3, 4, 5, 6, 7, 8, 9 or 10 restriction endonucleases. In certain embodiments, a set of restriction endonucleases is two restriction endonucleases. In certain embodiments, a set of restriction endonucleases is three restriction endonucleases. In certain embodiments, a set of restriction endonucleases is two restriction endonucleases and one of the restriction endonucleases is NlaIII. In some embodiments, one of the restriction endonucleases is NlaIII and the other restriction endonuclease is MboI or MseI. In certain embodiments, a set of restriction endonucleases is three restriction endonucleases and one of the restriction endonucleases is NlaIII. In some embodiments, a set of restriction endonucleases is three restriction endonucleases and one of the restriction endonucleases is NlaIII and another of the restriction endonucleases is either MboI or MseI. In some embodiments, the restriction endonucleases are NlaIII, MboI and MseI. Other restriction endonucleases and combinations of restriction endonucleases that enhance the preservation of spatial-proximal contiguity information are encompassed by the methods described herein.

In some embodiments, the restriction enzymes result in the same overhanging sequence. Examples of such enzymes include: AciI, HinP1I, HpalI, HpyCH4IV, MspI, and TaqI—all of which have 3′-CG-5′ overhangs on the 5′ end of the negative DNA strand. Similarly, BfaI, MseI, and CviQI have 3′-TA-5′ overhangs on the 5′ end of the negative DNA strand.

In some embodiments, the restriction enzymes result in different overhanging sequences.

In some embodiments, contact and digestion of DNA molecules with the two or more restriction endonucleases is performed at one time, i.e., simultaneously. In certain embodiments, the resultant spatial-proximal digested ends of the DNA molecules are then contacted with ligase to generate ligation junctions.

In certain embodiments, contact and digestion with the two or more restriction endonucleases is performed sequentially. In some embodiments, each sequential contact and digestion event can be with one or more restriction endonucleases. For example, a contact and digestion event could be a co-digestion with two restriction endonucleases. In some embodiments, the sequential contact and digestion with the two or more restriction endonucleases is performed in a defined order based on the particular restriction endonucleases used. In certain embodiments, at the conclusion of the sequential digestions (whether ordered or not) the resultant spatial-proximal digested ends of the DNA molecules are contacted with ligase to generate ligation junctions.

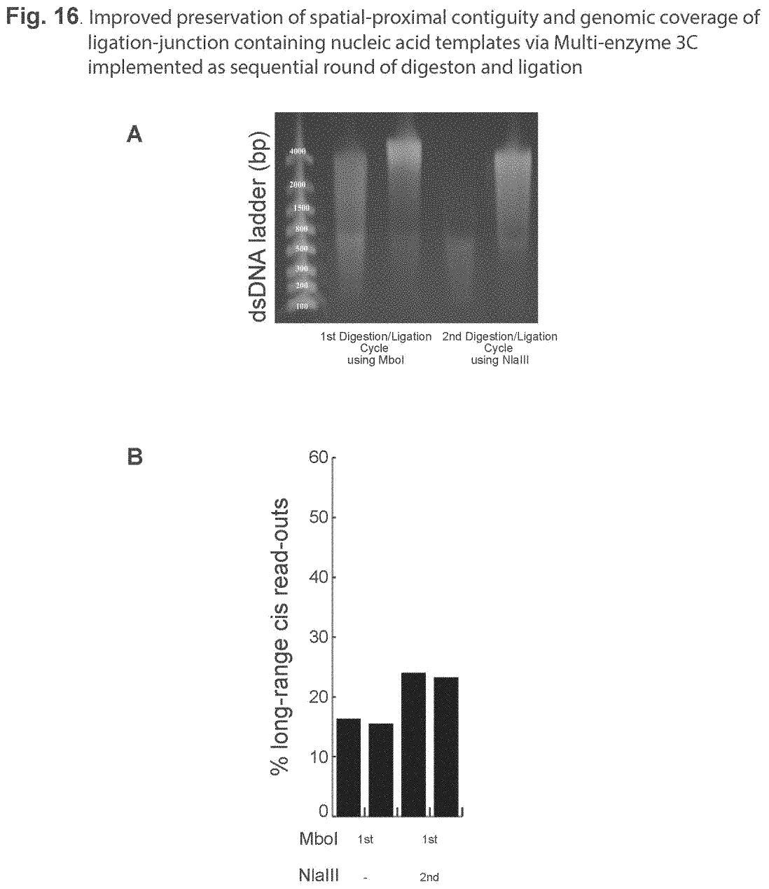

In certain embodiments, contact and digestion with each restriction endonuclease or combination of restriction endonucleases is performed sequentially and after the conclusion of each digestion event by one or more restriction endonucleases the resultant spatial-proximal digested ends of the DNA molecules are contacted with ligase to generate ligation junctions (see Example 8 and FIGS. 16A and 16B). The next digestion event in the sequence is performed with one or more different restriction endonucleases and upon the conclusion of digestion the spatial-proximal digested ends of the DNA molecules are contacted with ligase to generate further ligation junctions. In some embodiments, sequential digestion/ligation can be repeated 2, 3, 4, 5, 6 or more times. In certain embodiments multiple restriction endonuclease digestion/ligation steps are carried out in a defined order based on the particular restriction endonucleases used.

In certain embodiments, optimized 3C methods encompass other combinations of restriction endonucleases, types of overhanging ends produced (the same, different or a mixture of the same and different), simultaneous or sequential digestion, order of restriction endonucleases, the number of restriction endonucleases at each sequential step and whether ligation is performed once at the conclusion of all digestions or more frequently following each sequential digestion that improve the preservation of spatial-proximal contiguity information and/or the genome coverage of molecules comprising ligation junctions.

Size Selection

In some embodiments, proximity-ligated DNA molecules produced by using two or more restriction endonucleases are enriched for molecules containing ligation junctions that preserve spatial-proximal contiguity. In certain embodiments, enrichment is by size selection. In some embodiments, size selection is for larger fragments having sizes of approximately >5 kb, >10 kb, >20 kb, >30 kb, >40 kb, >50 kb, or >60 kb. Size selection can be carried out by any means known in the art.

In some embodiments, size selection is performed directly after reversal of cross-linking (if proximity-ligated molecules are crosslinked). In certain embodiments, size selection can be by gel extraction using manual or automated methods (e.g. Sage Science BluePippin instrument (Beverly, Mass.) or, using size selective DNA precipitation based methods (e.g. Circulomics Short Read Eliminator kits (Baltimore, Md.)).

In some embodiments, size selection is carried out following fragmentation of proximity-ligated molecules. In certain embodiments, size selection employs magnetic beads coated with carboxyl groups that bind DNA nonspecifically and reversibly, e.g., solid phase reversible immobilization (SPRI) beads, such Ampure Beads (Beckman Coulter; Brea, Calif.). In certain embodiments, the ratio of beads to sample volume can be adjusted to select larger fragments. For example, the ratio can be 0.4× to 0.8× or 0.4×, 0.5×, 0.6×, 0.7×, or 0.8×.

In some embodiments, size selection is carried out during library preparation, for example before or after performing PCR. A variety of size selection means are applicable, including the use of SPRI beads. Size selection of the described methods that is performed prior to construction of a library is not directed to optimization for molecules of a certain size for use with a particular sequencing machine. Rather, size selection as utilized in the described methods is directed to the purpose of enhancing data composition by impacting the proportion of templates containing ligation junctions and preserving spatial-proximal contiguity. For example, a maximum average library insert size of 350-450 bp is recommended for a HiSeq instruments compared to the much larger recommended insert size of ˜700 bp for optimized 3C.

In some embodiments, an optimized 3C protocol can have no size selection step or can have a single size selection step, two size selection steps or three size selection steps.

In certain embodiments, the means utilized for size selection, the size range selected and the applicability of using more than one size selection step can be evaluated for their effect on improving the preservation of spatial-proximal contiguity information by examining the percent of template molecules that represent long-range cis molecules, for example.

Optimization of a 3C method to improve the preservation of spatial-proximal contiguity information can be by utilizing multiple restriction endonucleases or multiple restriction endonucleases and size selection. Any of the described variations of multiple restriction endonuclease digestion can be utilized alone or in combination with any of the described variations of size selection. For example, a very rigorous size-selection following fragmentation of proximity-ligated molecules using a ratio of 0.4×SPRI beads to sample volume could be combined with sequential rounds of co-digestion and ligation.

In some embodiments, optimized 3C methods as described herein result in proximity-ligated DNA molecules that are derived from sequences covering essentially an entire genome.

In some embodiments, DNA molecules are obtained from any sample type where the nuclear architecture can remain intact. In some embodiments, DNA molecules are obtained from a sample selected from nuclei, cells, tissues, cell lines, primary cells, dissociated tissues, ground tissues, formalin-fixed paraffin-embedded (FFPE) samples, FFPE tissue sections or frozen tissue sections, deeply formalin-fixed samples or cell-free DNA. In certain embodiments, the sample is in an aqueous solution. In certain embodiments, the sample is affixed to a solid surface such as a slide. In certain embodiments, the sample is in an aqueous solution. In some embodiments, FFPE tissue is analyzed on a slide. In some embodiments, FFPE tissue removed from a slide (e.g., scrapped off physically, or by using laser capture microdissection) is analyzed. In some embodiments, frozen tissue is analyzed on a slide. In some embodiments, frozen tissue removed from a slide (e.g., scrapped off) is analyzed.

In some embodiments, the DNA molecules are obtained from a single cell, are obtained from two or more cells or are obtained from a tissue sample or a specific portion of a tissue sample. In some embodiments, the DNA molecules of a sample comprise two or more genomes or portions thereof.

In some embodiments, prior to preparation of a library for sequencing the proximity-ligated DNA molecules comprising ligation junctions are purified. In certain embodiments, if a sample was crosslinked, proximity-ligated DNA molecules comprising ligation junctions are contacted with a reagent that reverses crosslinking.

In some embodiments, a library of template molecules for DNA sequencing is prepared from proximity-ligated DNA molecules produced by the optimized 3C methods described herein.

In certain embodiments, the optimized 3C method include one or more steps specific to a 4C, 5C, Capture-C, 3C-ChIP (3C proximity ligation followed by ChIP-seq) or Methyl-3C method.

In some embodiments, a library of template molecules for DNA sequencing is prepared from the product of an optimized 3C method that include one or more steps 4C, 5C, Capture-C, 3C-ChIP or Methyl-3C method.

In some embodiments, a library of template molecules is sequenced to generate sequence reads comprising sequence information reflecting the use of 3C (3C-seq). In some embodiments, a library of template molecules is sequenced to generate sequence reads comprising sequence information that reflects the use of a 4C, 5C, Capture-C, 3C-ChIP or Methyl-3C method.

In certain embodiments, the sequencing is short-read sequencing. In certain embodiments, the optimized 3C method described herein result in at least 30%, at least 40%, at least 50% or at least 60% of the nucleic acid templates that are used to prepare a library for short-read sequencing being long-range cis molecules.

In some embodiments, prior to the preparation of a library that is used for short-read sequencing the proximity-ligated DNA molecules are fragmented to generate fragments of proximity-ligated DNA molecules comprising fragments spanning the ligation junctions.

In certain embodiments, the sequencing is long-read sequencing.

In some embodiments, a library of template molecules prepared by utilizing an optimized 3C protocol and one or more steps specific to a 4C, 5C, Capture-C, 3C-ChIP or Methyl-3C method, as described herein, is sequenced to generate sequence reads comprising sequence information. In certain embodiments, the sequencing is short-read sequencing. In certain embodiments, the sequencing is long-read sequencing.

Library preparation, sequencing and analysis of sequence information are as previously described herein.

In some embodiments, sequence information is utilized in applications that analyze spatial-proximal contiguity. In certain embodiments, sequence information is utilized for detection of pairwise 3D genome interactions of a genome or portion thereof. In certain embodiments, the 3D genome interaction is between promoters, enhancers, gene regulatory elements, GWAS loci, chromatin loop and topological domain anchors, repetitive elements, polycomb regions, gene bodies, exons or integrated viral sequences. In certain embodiments, sequence information is utilized for protein factor location analysis and 3D conformation analysis of a genome or portion thereof. In certain embodiments, protein factor location analysis and 3D conformation analysis comprises 3C-ChIP.

In some embodiments, optimized 3C methods are utilized in applications that benefit from increased coverage uniformity of read-pairs containing ligation junctions. In certain embodiments, sequence information is utilized for clustering and ordering of contigs of a genome or portion thereof. In certain embodiments, sequence information includes sequence information for each contig that is clustered and ordered. In certain embodiments, sequence information is utilized for clustering, ordering and orientating contigs of a genome or portion thereof. In some embodiments, sequence information is utilized for haplotype phasing of the genome or portion thereof. In some embodiments, sequence information is utilized for metagenome assemblies.

In some embodiments, sequence information is utilized in applications that depend on 1D genome coverage. In certain embodiments, sequence information is utilized for genomic rearrangement analysis of the genome or portion thereof. In certain embodiments, genomic rearrangement analysis comprises identification of a breakpoint. In certain embodiments, sequence information of a given sequence read is located upstream and downstream of the breakpoint. In certain embodiments, sequence information is utilized for DNA methylation analysis of a genome or portion thereof. In certain embodiments, sequence information is utilized for single nucleotide variant (SNV) discovery of a genome or portion thereof. In certain embodiments, sequence information is utilized for base polishing of long-range sequencing information of a genome or portion thereof. In certain embodiments, sequence information is utilized for highly sensitive copy number variation (CNV) analysis of a genome or portion thereof. In certain embodiments, a copy number variation (CNV) is an amplification. In certain embodiments, a copy number variation (CNV) is a heterozygous or homozygous deletion.

In certain embodiments, sequence information is utilized for variant discovery, haplotype phasing and genome assembly of a genome or portion thereof. In certain embodiments, sequence information is utilized for variant discovery and haplotype phasing in a first sample comprising a paternal genome and a second sample comprising a maternal genome and the phased variants of the paternal genome and the maternal genome are used to analyze sequence data of a fetal genome obtained from cfDNA of the mother. In certain embodiments, sequence information is utilized for haplotype phasing and genome assembly of a genome or portion thereof. In certain embodiments, sequence information is utilized for genome assembly and 3D conformation analysis of a genome or portion thereof. In certain embodiments, sequence information is utilized for DNA methylation analysis and detection of 3D genome interactions of a genome or portion thereof. In certain embodiments, sequence information is utilized for genome assembly and detection of 3D genome interaction of a genome or portion thereof.

In some embodiments, molecular contiguity information of proximity-ligated DNA molecules is preserved in addition to the spatial-proximal contiguity information preserved in ligation junctions. In certain embodiments, barcodes are used to preserve molecular contiguity information. In certain embodiments, barcodes are introduced into the proximity-ligated DNA molecules by contacting proximally-ligated DNA with a barcoded transposome linked bead prior to library preparation. In certain embodiments, the sequence information is utilized for detection of higher-order 3D genome interactions of a genome or portion thereof, by leveraging the preserved molecular contiguity of proximity-ligated DNA molecules. In certain embodiments, the sequence information is utilized for detection of three or more concurrent 3D genome interactions of the genome or portion thereof, by leveraging the preserved molecular contiguity of proximity-ligated DNA molecules. In certain embodiments, the sequence information is utilized for detection of virtual pairwise 3D genome interactions by leveraging the preserved molecular contiguity of proximity-ligated DNA molecules. In certain embodiments, a virtual pairwise 3D genome interaction is between restriction fragments that are not directly ligated to one another within a given proximity-ligated DNA molecule of the genome or portion thereof.

In certain embodiments, the pairwise interactions, virtual pairwise interactions, and/or higher order interactions obtained by leveraging the preserved molecular contiguity of proximity ligated DNA molecules is utilized for 3D genome interactions of the genome or portion thereof, genomic rearrangement analysis of the genome or portion thereof, clustering and ordering of contigs of the genome or portion thereof, determining contig orientation of the genome or portion thereof, haplotype phasing of the genome or portion thereof, DNA methylation analysis of the genome or portion thereof, single nucleotide variant (SNV) discovery of the genome or portion thereof, base polishing of long-range sequencing information of the genome or portion thereof, highly sensitive copy number variation (CNV) analysis of the genome or portion thereof or combinations thereof.

Single-Cells

In some embodiments, an optimized 3C protocol is to obtain sequence information from a single cell which provides a single cell profile.

Single-Cell 3C Via Cell/Nuclei Sorting (Either Before or after 3C) (“Plate” Method)

In some embodiments, in situ 3C proximity ligation is carried out “in bulk” (i.e. in a population of cells). Cells/nuclei are sorted using a cell sorting instrument (e.g. FACS and FANS), or manually, into discrete physical compartments such as wells of a microtiter plate. DNA is purified and amplified from each single cell using methods of whole genome amplification known in the art, such as multiple displacement amplification (MDA), or other means. Such an approach is analogous to Flyamer et al. Nature, 544(7648), pp. 110-114 (2017) or Tan et al. Science, 361(6405), pp. 924-928 (2018). Libraries are produced from amplified DNA molecules of each cell/nucleus. Libraries are sequenced and sequence reads are examined to obtain sequence information at single cell resolution.

In some embodiments, more pairwise interactions per cell may be captured by preserving the molecular contiguity of each proximally-ligated DNA molecule from each single cell. In certain embodiments, barcoded transposome linked beads (e.g. TELL-seq beads, Universal Sequencing Technologies, Carlsbad, Calif.) are applied to the purified proximally-ligated DNA in each microwell. Once the transposome-linked beads are applied, libraries are constructed for each individual cell. Reconstruction of the proximally-ligated DNA molecules from each single cell has the potential to dramatically improve the number of pairwise contacts per cell using the concept of “virtual pairs”, which means that 10 restriction fragment ligated together in a ligation product would conventionally be derived from ˜9 ligation junctions and produce 9 pairwise 3D contacts. If the entire 10 fragments on a given ligation product were revealed, this would inform 45 total combinations of pairwise 3D contacts ((10*9)/2), or the equation P=(((n*(n−1))/2), where P is the total number of pairwise 3D contacts obtained per ligation product, and n is the number of restriction fragment concatemerized into the ligation product. If 25 restriction fragments were in a ligation product, this would produce ˜24 pairwise contacts with traditional library prep, or 300 “virtual pairs” if the molecular contiguity of each 3C ligation product was preserved during library prep. This would represent a log-order increase in information content per cell.

Single-Cell 3C Via Droplet Microfluidics Approaches (“Droplet” Method)

In some embodiments, in situ 3C proximity ligation is carried out “in bulk” (i.e. in a population of cells). Cells/nuclei are input into a commercial (e.g. 10× Genomics (Pleasanton, Calif.), Bio-Rad, (Hercules, Calif.), Mission Bio (South San Francisco, Calif.) or homebrew (e.g. Drop-Seq) droplet microfluidics system where reagents are delivered to barcode and amplify proximally-ligated DNA from each single cell/nucleus. Libraries are produced from amplified DNA molecules of each cell/nucleus. Libraries are sequenced and sequence reads are examined to obtain sequence information at single cell resolution.

In some embodiments, 4C is utilized for library preparation (single-cell 4C). For 4C in the plate and droplet single cell methods, targeted amplification with a locus specific primer pair (which is what is done in 4C) comprising cell barcodes rather than whole genome amplification is carried out.

In some embodiments, Capture C is used to enrich for specific targets (templates are enriched by target enrichment and sequenced). Since the templates have the cell barcode(s) based on the protocol used to obtain single cells (see above) the sequence information can be assigned to a single cell.

Spatial Positioning (“Spatial” Method)

In some embodiments, analysis of tissue sections processed using an optimized 3C protocol (or HiC protocol) can provide spatial positioning for sequence information obtained from portions of the tissue section or from single cells. In certain embodiments, in situ 3C (or HiC) proximity ligation is carried out while the tissue is held intact on a surface such as a slide, and then the tissue (now comprised of proximally-ligated nuclei) is micro-dissected into spatially distinct regions. In some embodiments, a spatially distinct region is a grid (e.g. 8×12) sometimes having quadrants, concentric circles (like a bulls eye), peripheral tumor cells that contact non-tumor cells or the tumor microenvironment, cell clusters in sub-regions of a tissue, or a collection of single cells. Each spatially distinct region can be treated as its own “sample” and processed as a distinct physical collection of cells or single-cells can be obtained according to the examples above and processed individually. In certain embodiments, a tissue section is first micro-dissected into spatially distinct regions and each spatially distinct region is treated as its own in situ 3C (or HiC) proximity ligation reaction and processed as a distinct physical collection of cells or single-cells can be obtained according to the examples above and processed individually. During the data analysis phase, tissue 3C (or HiC) profiles of spatially distinct regions or single cell 3C (or HiC) profiles can be attributed to their spatial positioning within a tissue section.

In certain embodiments, each spatially distinct region may not need to be treated as its own separate in situ 3C (or HiC) reaction. In certain embodiments, methods similar to MULTI-seq (McGinnis et al. Nature methods, 16(7), p. 619 (2019)) can be adapted for sample barcoding in the context of single cell 3C (or HiC) analysis. For example, cells/nuclei can be collected from each spatially defined region from a tissue section. The samples would then be reacted with lipid-modified oligonucleotide (LMO) or cholesterol-modified oligonucleotide (CMO), which imbeds into the plasma membrane of a cell membrane or nuclear membrane. The oligonucleotide would comprise a means to be amplified after the proximally-ligated nuclei are partitioned into wells of a plate or droplets. During the data analysis phase, the single cell 3C (or HiC) profiles can be attributed to their spatial positioning within a tissue section, and the co-amplified sample barcode sequence corresponding to each single cell would serve as the sample identifier that was introducing during the sample tagging reaction.

In some embodiments, 4C is utilized in the analysis is of tissue section. Targeted amplification is carried out with a locus specific primer pair using the 3C templates that are produced from each spatially defined region that is micro-dissected from the tissue section.

Library Prep Manipulations

In some embodiments, the above-described 3C methods are combined with target enrichment methods. In certain embodiments, target enrichment is PCR based.

Post Library Prep Manipulations

In some embodiments, the above-described 3C methods are combined with target enrichment methods. In certain embodiments, target enrichment is probe based. In certain embodiments, target enrichment is PCR based.

In some embodiments, Capture C is used to enrich for specific targets (templates are enriched by target enrichment and sequenced).

Kits

In some embodiments, provided are kits for carrying out methods described herein. Kits often comprise one or more containers that contain one or more components described herein. A kit comprises one or more components in any number of separate containers, packets, tubes, vials, multiwell plates and the like, or components may be combined in various combinations in such containers. Kit components and reagents are as described herein.

HiC Kits

In some embodiments, a kit comprises one or more of (a) three or more restriction endonucleases; (b) a restriction endonuclease buffer; and (c) one or more of a biotinylated nucleotide, unlabeled nucleotides, a DNA polymerase, ligase, ligase buffer, one or more additional buffers and reagents for reversing cross-linking.

In some embodiments, a kit comprises one or more of (a) four restriction endonucleases; (b) a restriction endonuclease buffer; and (c) one or more of a biotinylated nucleotide, unlabeled nucleotides, a DNA polymerase, ligase, ligase buffer, one or more additional buffers and reagents for reversing cross-linking. In certain embodiments, the four restriction endonucleases are: MboI, HinfI, MseI and DdeI. In certain embodiments, the four restriction endonucleases are: HpyCH4IV, HinfI, HinP1I and MseI.

In some embodiments, a kit comprises one or more of: four restriction endonucleases; (b) two or more restriction endonuclease buffers; and (c) one or more of a biotinylated nucleotide, unlabeled nucleotides, a DNA polymerase, ligase, ligase buffer, one or more additional buffers and reagents for reversing cross-linking. In some embodiments, the two or more restriction endonuclease buffers are in separate containers from the four restriction endonucleases. In some embodiments, each restriction endonuclease has a theoretical digestion frequency of at least 1 in 256. In some embodiments, at least two of the restriction endonucleases require unique buffers for high level activity.

In some embodiments, the restriction endonucleases are in separate containers. In some embodiments, the restriction endonucleases are in a single container. In some embodiments, each restriction endonuclease has a high activity level in a common restriction endonuclease buffer and each restriction endonuclease has a theoretical digestion frequency of at least 1 in 256. In some embodiments, the restriction endonuclease buffer is in a separate container from the restriction endonucleases.

3C Kits

In some embodiments, a kit comprises one or more of (a) two or more restriction endonucleases; (b) a restriction endonuclease buffer; and (c) one or more of ligase, ligase buffer, one or more additional buffers and reagents for reversing cross-linking, one or more additional buffers and reagents for size selection, a bead-linked transposome, primers with barcode oligonucleotides, one or more reagents to create a sequencing library and does not include a biotinylated nucleotide or a labelled nucleotide.

In some embodiments, a kit comprises one or more of (a) two restriction endonucleases; (b) a restriction endonuclease buffer; and (c) one or more of ligase, ligase buffer, one or more additional buffers and reagents for reversing cross-linking, one or more additional buffers and reagents for size selection, a bead-linked transposome, primers with barcode oligonucleotides, one or more reagents to create a sequencing library and does not include a biotinylated nucleotide or a labelled nucleotide. In certain embodiments, one of the restriction endonucleases is NlaIII. In certain embodiments, one of the restriction endonucleases is NlaIII and the other restriction endonuclease is MboI or MseI.

In some embodiments, a kit comprises one or more of (a) three restriction endonucleases; (b) one or more of restriction endonuclease buffers; and (c) one or more of ligase, ligase buffer, one or more additional buffers and reagents for reversing cross-linking, one or more additional buffers and reagents for size selection, a bead-linked transposome, primers with barcode oligonucleotides, one or more reagents to create a sequencing library and does not include a biotinylated nucleotide or a labelled nucleotide. In certain embodiments, one of the restriction endonucleases is NlaIII. In certain embodiments, one of the restriction endonucleases is NlaIII and one of the other restriction endonucleases is MboI or MseI. In certain embodiments, the restriction endonucleases are: NlaIII, MboI and MseI.

In some embodiments, the restriction endonucleases of a kit produce the same overhanging sequence. In some embodiments, the restriction endonucleases of a kit produce different overhanging sequences. In some embodiments, digestion with the two or more restriction endonucleases of a kit can be carried out at the same time. In some embodiments, digestion with two or more restriction endonucleases of a kit cannot be carried out at the same time.

In some embodiments, the restriction endonucleases of a kit are in separate containers. In some embodiments, the restriction endonucleases of a kit are in a single container. In some embodiments, the restriction endonucleases of a kit are in more than one container and at least one container contains more than one restriction endonuclease. In some embodiments, each restriction endonuclease of a kit has a high activity level in a common restriction endonuclease buffer and the buffer is in one container. In some embodiments, more than one buffer is in a kit and the buffers are in separate containers. In some embodiments, a restriction endonuclease buffer is in a separate container from a restriction endonuclease.

In certain embodiments, the kit comprises instructions. In some embodiments, the instructions recite the order that the restriction enzymes of a kit are to be used.

A kit sometimes is utilized in conjunction with a process, and can include instructions for performing one or more processes and/or a description of one or more compositions. A kit may be utilized to carry out a process described herein. Instructions and/or descriptions may be in tangible form (e.g., paper and the like) or electronic form (e.g., computer readable file on a tangle medium (e.g., compact disc) and the like) and may be included in a kit insert. A kit also may include a written description of an internet location that provides such instructions or descriptions.

Libraries

In some embodiments, libraries are constructed as described herein based on the use of HiC or optimized 3C methods.

### EXAMPLES

The examples set forth below illustrate certain embodiments and do not limit the technology.

### Example 1: Selection of Optimal RE

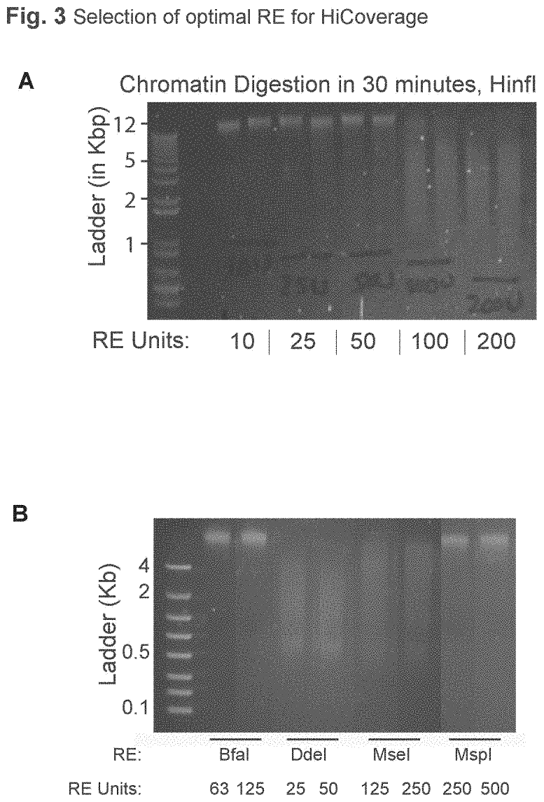

FIGS. 3A to 3B show the chromatin digestion efficiency of candidate RE that may be used in conjunction with MboI to increase RE cut site density and genome coverage. Criteria for selection included that the REs must have 100% activity levels in a common RE digest buffer. RE must also be commercially available at a high enough concentration such that a reasonable volume of each enzyme can be utilizing during HiC. Lastly, the combination of RE must maximizing the in silico digestion frequency (each enzyme has a theoretical digestion frequency of at least 1 in 256). These criteria would help insure the biochemical compatible, efficiency, and practicality of the RE combination in HiC context, and, deliver maximum genome coverage.

Crosslinked GM19240 cells were digested with increasing amounts of HinfI for 30 min, in replicate. After digestion, crosslinks were reversed, DNA was purified, and gel electrophoresis was performed. At least 100 U of HinfI were required for efficient chromatin digestion, evidenced by the smaller molecular weight of the digested DNA sample. Because HinfI can reach efficiency levels of crosslinked chromatin digestion with a reasonable amount of RE units (e.g., 100 units), and is compatible with the same buffer as MboI, HinfI can be used in conjunction with MboI (see FIG. 3A). Both MboI and Hinf1 efficiently cleave in CutSmart™ Buffer (New England Biolabs, Beverly, Mass.) (1× −50 mM Potassium acetate, 20 mM Tris-acetate, 10 mM Magnesium acetate, 100 ug/ml BSA, pH 7.9 at 25° C.).

To select additional RE to further increase coverage uniformity, 4 additional 4-cutters (BfaI, DdeI, MseI, and MspI) with 100% reported activity levels in a RE buffer that is also compatible with MboI and HinfI (CutSmart™ Buffer) were identified. Crosslinked GM12878 cells were digested with a maximum practical amount of each enzyme, in replicate. After digestion, crosslinks were reversed, DNA was purified, and gel electrophoresis was performed. Surprising, despite the reasonable RE concentrations, buffer compatibility, and in silico cut site frequency (1 in 256), only 2/4 of the RE showed efficient RE digestion during HiC (see FIG. 3B). These 2 REs DdeI (at least 25 units) and MseI (at least 125 units) were selected as REs to be used in conjunction with MboI (at least 100 units) and HinfI (at least 100 units) to reach optimal RE cut site density and genome coverage. However, when crosslinked GM 12878 cells were simultaneously digested with these four enzymes and post-digestion fragment size was examined by gel electrophoresis, surprisingly the post-digestion fragment size was comparable to that of a single enzyme (data not shown). This suggested that not every cut site is being cut, even using a combination of four enzymes, and that it could not be predicted that sequence coverage adjacent to each could be obtained so as to achieve full genome coverage.

### Example 2: SNV Discovery

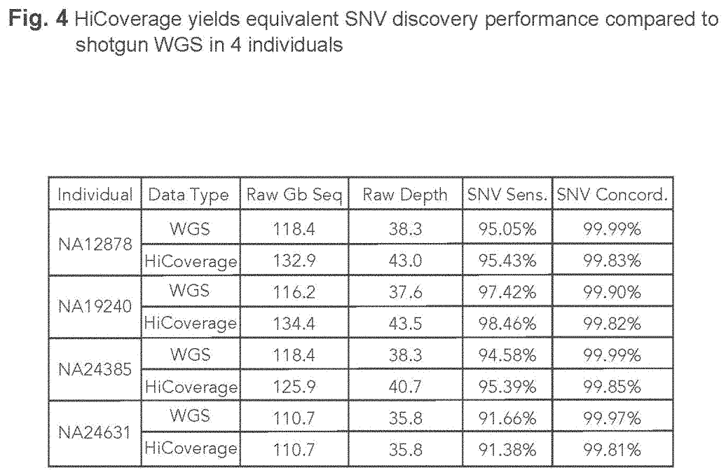

FIG. 4 shows how the improved genome coverage from HiCoverage enables highly sensitive SNV discovery, and is comparable to shotgun WGS. For this analysis, the raw, 2×150 bp HiC raw reads were aligned to the hg19 human genome using BWA mem with default parameters and including the −SP5M option, which aligns the read-pairs as single ends but retains the mate-pair information, and also retains the 5′ most alignment as the primary alignment for chimeric reads. After alignment, Read Groups were added using GATK and PCR duplicates removed using PicardTools. GATK was then used for Base Recalibration, and Print Reads, and then variants were called using GATK Haplotype Caller, and recalibrated using GATK Variant Recalibration with a non-default tranche value of 99.9 and a MaxGaussian setting of either 4 or 8. For the shotgun WGS data, we obtained the raw sequence data for NA12878, NA24385, and NA24631 from the Genome in a Bottle consortium (Zook, Scientific Data, 2016). For NA12878 and NA24385, the raw, 2×148 bp, read-pairs were sub-sampled such that the total depth was comparable to the donor-matched HiCoverage datasets. For NA24631, the entire available 2×250 bp datasets was downloaded and used for subsequent analyses. For the 4^{th} individual (NA19240), shotgun WGS data was downloaded from Steinberg et al. BioRxiv, p. 067447 (2016), and sub-sampled such that the total depth was comparable to the donor-matched HiCoverage datasets. After collecting and sampling the datasets as described above, the read-pairs were processed as described above for HiC, except during alignment the data were mapped as a true mate-pair (−M) and variant calls were recalibrated always using a default tranche value (99.0%) and default MaxGaussian (8). For all HiCoverage datasets, only bi-allelic homozygous or heterozygous SNVs on autosomes with a supporting read-depth minimum of 5 reads were retained for SNV sensitivity benchmarking analysis against a “truth” set of SNV calls from shotgun WGS data. For the GIAB genomes (NA12878, NA24385, NA24631), we further subset the variants for benchmarking analyses as only those in high confidence regions defined by the GIAB. For the truth set on variants from shotgun WGS data in the three GIAB genomes, we used bi-allelic homozygous or heterozygous SNVs on autosomes extracted from the same high confidence regions set forth by the GIAB consortium (Zook, Scientific Data, 2016). For NA19240, the truth variant calls were obtained from the 1000 Genomes Project.

### Example 3: HiCoverage of a Variety of Genomes

Twenty vertebrate genome assemblies, two plant genome assemblies, two insect genome assemblies, and two parasite genome assemblies were downloaded from various sources such as GenomeArk (https://vgp.github.io/genomeark/) for vertebrates and NCBI (https://www.ncbi.nlm.nih.gov/genome/) for other genomes. Genomes were then digested in silico using either the four restriction enzymes cut site motifs for MboI, MseI, DdeI, and HinfI, or, for just the single restriction enzyme MboI to mimic a relatively low density restriction enzyme method. To estimate the expected coverage, or what fraction of the genomic bases would be “visible” to HiC, the fraction of genomic bases that are within 250 bp from a restriction enzyme cut site was calculated. These fractions are plotted on the y-axis for each genome (x-axis labels) (FIG. 14A—vertebrate genomes; FIG. 14B—insect, plant and parasite genomes).

The results indicate that HiCoverage using a combination of restriction enzymes enables near complete genomic coverage across representative plant and animal species, and therefore various plant and animal species should be robust to the unique benefits of HiCoverage data described herein.

### Example 4: HiCoverage and Coverage Uniformity

Crosslinked GM12878 cells were subject to HiCoverage experiment using MboI, MseI, DdeI, and HinfI and sequenced to approximately 37× raw depth. Depth-matched low density HiC data using MboI in GM12878 cells were downloaded from Rao, Cell, 2014. Each dataset was mapped to the hg19 reference genome using bwa mem −SP5M and deduplicated using PicardTools. The genome coverage histograms were then generated using DeepTools. As illustrated in FIG. 15, the results show the drastic difference in observed coverage uniformity, with the coverage uniformity of the HiCoverage data dramatically improved relative to low density RE approaches.

### Example 5: Multi-Enzyme 3C—Simultaneous Digestion

Crosslinked GM12878 cells were digested with either one, two, or three restriction enzymes (denoted across categorical axis labels of FIG. 11) simultaneously, in duplicate, using either MboI, NlaIII, or MseI. After digestion, proximity ligation was performed using ligase. Then, crosslinks were reversed and proximally-ligated DNA was purified. Proximally-ligated DNA was then sheared and size selected using a 0.6× ratio of Ampure Beads to sample volume. Lastly, Illumina sequencing libraries were constructed, PCR amplified, and purified using a 0.6× ratio of Ampure Beads to sample volume. 3C libraries were sequenced on a MiniSeq yielding ˜1M raw PE reads per sample. After mapping and deduplication, the fraction of read-pairs that represent long-range (>15 kb insert size) intra-chromosomal interactions were enumerated and plotted along the y-axis for each permutation of restriction enzyme co-digestion conditions (see FIG. 11).

The sequencing results shown in FIG. 11 indicate that the implementation of certain restriction enzymes improve the preservation of spatial-proximal contiguity in the nucleic acid templates (when used in the context of size selection) The best results, of the restriction enzymes tested, derived from conditions that include NlaIII. Second, the use of two restriction enzymes improves the preservation of spatial-proximal contiguity in the nucleic acid templates relative to the use of a single enzyme (e.g., NlaIII+MboI or MseI vs. NlaIII alone). However, adding a third enzyme into the cocktail under these specific conditions (e.g., NlaIII+MboI+MseI or (NlaIII+MseI+MboI) does not further improve the preservation of spatial-proximal contiguity in the nucleic acid templates, but could however increase the coverage uniformity of ligation-junction containing nucleic acid templates.

### Example 6: Multi-Enzyme 3C—Sequential Digestion

Crosslinked GM12878 cells were digested with either one, two, or three restriction enzymes sequentially, in duplicate, using either MboI, NlaIII, or MseI. The order of restriction enzyme digestion is denoted as categorical axis labels (see FIG. 12). For example, in the case of a triple digestion (far right column in the bar plot of FIG. 12), GM12878 nuclei were first digested with NlaIII. After the NlaIII reaction was complete, the nuclei were then digested with MboI. After the MboI digestion was complete, the nuclei were then digested with MseI. After the MseI digestion was complete, proximal ligation was carried out using a ligase. Then, crosslinks were reversed and proximally-ligated DNA was purified. Proximally-ligated DNA was then sheared and size selected using a 0.6× ratio of Ampure Beads to sample volume. Lastly, Illumina sequencing libraries were constructed, PCR amplified, and purified using a 0.6× ratio of Ampure Beads to sample volume. 3C libraries were sequenced on a MiniSeq yielding ˜1M raw PE reads per sample. After mapping and deduplication, the fraction of read-pairs that represent long-range (>15 kb inserts) intra-chromosomal interactions were enumerated and plotted along the y-axis for each condition (see FIG. 12).

The sequencing results indicate that sample digestion with >1 restriction enzyme improve the preservation of spatial-proximal contiguity in the nucleic acid templates relative to digestion with a single enzyme. This result is surprising given that digestion with multiple restriction enzymes creates incompatible ends for proximity ligation, yet proximity ligation is still evidence by the increase in fraction of long-range cis read-outs. For example, sequential digestion with NlaIII and MseI, in either order, furthest improve the preservation of spatial-proximal contiguity in the nucleic acid templates. The sequencing results also indicate the order of sequential digestion appears to impact the sequencing results, (e.g., the condition starting with MseI and followed by NlaIII have the greatest preservation of spatial-proximal contiguity in the nucleic acid templates). However, similar to co-digestion results (FIG. 11), adding a third enzyme into the series of restriction digestions under these conditions did not further improve the preservation of spatial-proximal contiguity in the nucleic acid templates relative to two digestions, but could however increase the coverage uniformity of ligation-junction containing nucleic acid templates. Without being held to a theory, the failure of a third enzyme in this particular combination of restriction endonucleases to increase the preservation of spatial-proximal contiguity in the nucleic acid templates could be because of an increase in incompatible ends that cannot be proximally-ligated. As a possible means to overcome this problem, restriction enzymes could be used that produce the same overhanging sequence and therefore compatible for sticky end ligation in the 3C experiment. Another possible means to overcome this problem could be performing sequential rounds of digestion and ligation.

### Example 7: Size Selection of 3C Libraries

Crosslinked GM12878 cells were digested with NlaIII. After digestion, proximity ligation was performed using a ligase. Then, crosslinks were reversed and proximally-ligated DNA was purified. Proximally-ligated DNA was then sheared and split into 3 groups of DNA and subject to DNA size selection using either a 0.7×, 0.6×, or 0.5× ratio of Ampure Beads to sample volume, in quadruplicate. Illumina sequencing libraries were constructed using the 12 DNA samples and PCR amplified. After PCR amplification, 2 libraries from each group were purified using a 0.6× ratio of Ampure Beads to sample volume, with the other 2 libraries from each group were purified (and size selected) using a 0.8× ratio of Ampure Beads to sample volume. 3C libraries were sequenced on a MiniSeq yielding ˜1M raw PE reads per sample. After mapping and deduplication, the fraction of read-pairs that represent long-range (>15 kb insert size) intra-chromosomal interactions were enumerated and plotted along the y-axis for each permutation of post-shearing and post-PCR size selection conditions.

The sequencing results shown in FIG. 13 indicate the overall trend that libraries that have undergone size selection favored towards larger nucleic acid templates (i.e. the lowest ratios of Ampure beads to sample volume, right side of bar plot) show the greatest preservation of spatial-proximal contiguity in the nucleic acid templates. For example, when considering only the conditions that received the 0.8× post-PCR size selection, the fraction of long-range cis read-outs increases from 33%, to 36.5%, to 39%. This is because 0.8× is unlikely to have a size selection effect since it's a higher ratio than the lowest post-shearing size selection, meaning the post-shearing size selection parameters (and thus the molecular size of the nucleic acid templates) are driving the sequencing results.

### Example 8: Multi-Enzyme 3C—Sequential Rounds of Digestion and Ligation

Crosslinked GM12878 cells were subject to two consecutive rounds of digestion and proximity ligation reactions. In the first round, GM12878 nuclei were digested with MboI and then proximity ligation was performed using ligase. Then nuclei were pelleted and resuspended in 1× restriction digestion buffer (CutSmart). Nuclei were then subject to a second round of restriction digestion using NlaIII, and then subject to a second round of proximity ligation using a ligase. As a control, some nuclei were set aside after the first round of digestion and proximity ligation. Then, crosslinks were reversed in all nuclei samples and proximally-ligated DNA was purified. Proximally-ligated DNA was then sheared and size selected using a 0.7× ratio of Ampure Beads to sample volume. Lastly, Illumina sequencing libraries were constructed, PCR amplified, and purified using a 0.8× ratio of Ampure Beads to sample volume. 3C libraries were sequenced on a MiniSeq yielding ˜1M raw PE reads per sample. After mapping and deduplication, the fraction of read-pairs that represent long-range (>15 kb inserts) intra-chromosomal interactions were enumerated and plotted along the y-axis for each condition. Throughout the experiment, a small aliquot of nuclei was taken after each digestion and ligation reaction (4 aliquots total) in order to obtain the molecular size of DNA after each step. DNA is these aliquots of nuclei were obtained by crosslink reversal and DNA purification. DNA was then analyzed by gel electrophoresis using a FlashGel (Lonza) with a molecular weight ladder as indicated.

FIG. 16A shows gel electrophoresis results indicate that chromatin was being effectively digested by MboI and re-ligated, evidenced by the lower molecular weight of the digested chromatin, and increase in molecular weight after proximity ligation. The results also indicate the proximally-ligated chromatin was being effectively re-digested by NlaIII and re-ligated, evidenced by the lower molecular weight of the re-digested chromatin, and increase in molecular weight after the second round of proximity ligation. The sequencing results indicate that addition of a second sequential round of digestion and re-ligation can improve the preservation of spatial-proximal contiguity in the nucleic acid templates (see FIG. 16B), while simultaneously increasing the coverage uniformity of ligation-junction containing nucleic acid templates.

### Example 9: Non-Limiting Examples of Embodiments

A1. A method for preparing DNA molecules from a sample comprising:
(a) contacting cross-linked DNA molecules of a sample comprising a genome or portion thereof with a set of restriction endonucleases; thereby generating spatial-proximal digested ends of cross-linked DNA molecules;
(b) contacting the spatial-proximal digested ends of cross-linked DNA molecules with ligase, thereby generating cross-linked proximity-ligated DNA molecules comprising ligation junctions;
(c) contacting the cross-linked proximity-ligated DNA molecules with a reagent that reverses cross-linking, thereby generating proximity-ligated DNA molecules comprising ligation junctions; and
(d) fragmenting the proximity-ligated DNA molecules to generate fragments of proximity-ligated DNA molecules comprising fragments spanning the ligation junctions, wherein fragments spanning the ligation junctions and of lengths that can be templates for short range sequencing, comprise sequences of essentially the whole genome or portion thereof.

A2. The method of embodiment A1, wherein the fragments spanning the ligation junctions comprise fragments up 750 base pairs.

A3. The method of embodiment A1 or A2, wherein each restriction endonuclease of the set has a high activity level in a common buffer and each restriction endonuclease of the set has a theoretical digestion frequency of at least 1 in 256.

A4. The method of any one of embodiments A1 to A3, wherein the set of restriction endonucleases consists of four restriction endonucleases.

A5. The method of embodiment A4, wherein the restriction endonucleases are: MboI, HinfI, MseI and DdeI.

A5.1. The method of embodiment A4, wherein the restriction endonucleases are: HpyCH4IV, HinfI, HinP1I and MseI.

A6. The method of anyone of embodiments A1 to A5.1, wherein the DNA molecules are obtained from a sample selected from nuclei, cells, tissues, formalin-fixed paraffin-embedded (FFPE) samples, deeply formalin-fixed samples or cell-free DNA.

A7. The method of anyone of embodiments A1 to A5.1, wherein the DNA molecules are obtained from a single cell.

A7.1. The method of anyone of embodiments A1 to A5.1, wherein the DNA molecules are obtained from two or more cells.

A8. The method of any one of embodiments A1 to A5.1, wherein the cross-linked DNA molecules of a sample comprise two or more genomes or portions thereof.

A9. The method of anyone of embodiments A1 to A8, wherein the proximity-ligated DNA molecules are analyzed in a chromatin conformation assay.

A10. The method of embodiment A9, wherein the chromatin conformation assay is Capture-C, 3C, 4C, 5C, HiC, Capture-HiC, HiChIP, PLAC-seq, tethered chromosome capture (TCC), HiCulfite, Methyl-HiC, HiChIRP or combinations thereof.

A11. The method of embodiment A9, wherein the assay is genome-wide.

A11.1. The method of embodiment A11, wherein the assay is 3C, HiC, tethered chromosome capture (TCC), HiCulfite, Methyl-HiC or combinations thereof.

A12. The method of embodiment A9, wherein the assay is directed to one or more target regions in the genome.

A12.1. The method of embodiment A12, wherein the assays is Capture-C, 4C, 5C, Capture-HiC, HiChIP, PLAC-seq, HiChIRP or combinations thereof.

A13. The method of embodiment A12, wherein the targets are single nucleotide variations, insertions, deletions, copy number variations, genomic rearrangements or targets for phasing.

A14. The method embodiment A12 or A13, wherein the sample comprises a cancer genome and the target region is associated with a phenotype.

A15. The method of any one of embodiments A1 to A14, wherein the fragments of the proximity-ligated DNA molecules comprising fragments spanning the ligation junctions are used to prepare a library of template molecules for DNA sequencing.

A15.1. The method of embodiment A15, wherein the ligation junctions are marked with an affinity purification marker.

A15.2 The method of embodiment A15.1, wherein the affinity purification marker is biotin conjugated to a nucleotide.

A15.3. The method of embodiment A15.2, whereby enrichment is by affinity purification of the affinity purification marker with an affinity purification molecule.

A16. The method of embodiment A15.3, wherein fragments spanning the ligation junctions are enriched to prepare a library of template molecules for DNA sequencing.

A17. The method of any one of embodiments A15 to A16 that are used is in a HiC, Capture-HiC, HiSCIP, PLAC-seq, HiCulfite or Methyl-HiC method.

A17.1. The method of embodiment A15.3, wherein the affinity purification molecule is streptavidin.

A17.2. The method of embodiment A16, where enrichment for fragmented proximity-ligated DNA molecules comprising ligation junctions is by size selection.

A18. The method of any one of embodiments A15 to A17.2, wherein the library of template molecules provides uniform genome-wide coverage of a genome or portion thereof.

A18.1. The method of any one of embodiments A15 to A18, wherein the library of template molecules is sequenced to generate sequence reads comprising sequence information.

A19. The method of embodiment A18.1, wherein the sequencing is short read sequencing.

A20. The method of embodiment A18.1 or A19, wherein the sequence information is utilized for genomic rearrangement analysis of the genome or portion thereof.

A21. The method of embodiment A20, wherein the genomic rearrangement analysis comprises identification of a breakpoint.

A22. The method of embodiment A21, wherein sequence information of a given sequence read is located upstream and downstream of the breakpoint.

A23. The method of embodiment A18.1 or A19, wherein the sequence information is utilized for clustering and ordering of contigs of the genome or portion thereof.

A24. The method of embodiment A23, wherein sequence information includes sequence information for each contig that is clustered and ordered.

A25. The method of embodiment A18.1 or A19, wherein the sequence information is utilized to determine contig orientation of the genome or portion thereof.

A26. The method of embodiment A18.1 or A19, wherein the sequence information is utilized for clustering, ordering and orientating contigs of the genome or portion thereof.

A27. The method of embodiment A18.1 or A19, wherein the sequence information is utilized for detection of pairwise 3D genome interactions of the genome or portion thereof.

A28. The method of embodiment A27, wherein the 3D genome interaction is between promoters, enhancers, gene regulatory elements, GWAS loci, chromatin loop and topological domain anchors, repetitive elements, polycomb regions, gene bodies, exons or integrated viral sequences.

A29. The method of embodiment A18.1 or A19, wherein the sequence information is utilized for protein factor location analysis and 3D conformation analysis of the genome or portion thereof.

A30. The method of embodiment A29, wherein the protein factor location analysis and 3D conformation analysis comprises PLAC-seq or HiChIP.

A31. The method of embodiment A18.1 or A19, wherein the sequence information is utilized for haplotype phasing of the genome or portion thereof.

A32. The method of embodiment A18.1 or A19, wherein the sequence information is utilized for genome assembly and 3D conformation analysis of the genome or portion thereof.

A33. The method of embodiment A18.1 or A19, wherein the sequence information is utilized for DNA methylation analysis of the genome or portion thereof.

A33.1. The method of embodiment A18.1 or A19, wherein the sequence information is utilized for DNA methylation analysis and detection of 3D genome interactions of the genome or portion thereof.

A34. The method of embodiment A18.1 or A19, wherein the sequence information is utilized for single nucleotide variant (SNV) discovery of the genome or portion thereof.

A35. The method of embodiment A18.1 or A19, wherein the sequence information is utilized for base polishing of long-range sequencing information of the genome or portion thereof.

A36. The method of embodiment A18.1 or A19, wherein the sequence information is utilized for highly sensitive copy number variation (CNV) analysis of the genome or portion thereof.

A37. The method of embodiment A36, wherein the copy number variation (CNV) is an amplification.

A38. The method of embodiment A36, wherein the copy number variation (CNV) is a heterozygous or homozygous deletion.

A39. The method of embodiment A18.1 or A19, wherein the sequence information is utilized for variant discovery, haplotype phasing and genome assembly of the genome or portion thereof.

A39.1 The method of embodiment A18.1 or A19, wherein the sequence information is utilized for variant discovery and haplotype phasing in a first sample comprising a paternal genome and a second sample comprising a maternal genome and the phased variants of the paternal genome and the maternal genome are used to analyze sequence data of a fetal genome obtained from cfDNA of the mother.

A40. The method of embodiment A18.1 or A19, wherein the sequence information is utilized for haplotype phasing and genome assembly of the genome or portion thereof.

A41. The method of embodiment A18.1 or A19, wherein the sequence information is utilized for genome assembly and detection of 3D genome interaction of the genome or portion thereof.

B1. A method for preparing DNA molecules from a sample comprising:
(a) contacting cross-linked DNA molecules of a sample comprising a genome or portion thereof with a first restriction endonuclease, thereby generating first spatial-proximal digested ends of cross-linked DNA molecules;
(b) contacting the first spatial-proximal digested ends of cross-linked DNA molecules with ligase, thereby generating first cross-linked proximity-ligated DNA molecules comprising first ligation junctions;
(c) contacting the first cross-linked proximity-ligated DNA molecules comprising first ligation junctions with a second restriction endonuclease, thereby generating second spatial-proximal digested ends of cross-linked DNA molecules;
(d) contacting the second spatial-proximal digested ends of cross-linked DNA molecules with ligase, thereby generating second cross-linked proximity-ligated DNA molecules comprising first and second ligation junctions;
(e) contacting the second cross-linked proximity-ligated DNA molecules comprising first and second ligation junctions with a third restriction endonuclease, thereby generating third spatial-proximal digested ends of cross-linked DNA molecules;
(f) contacting the third spatial-proximal digested ends of cross-linked DNA molecules with ligase, thereby generating third cross-linked proximity-ligated DNA molecules comprising first, second and third ligation junctions;
(g) contacting the third cross-linked proximity-ligated DNA molecules comprising first, second and third ligation junctions with a fourth restriction endonuclease, thereby generating fourth spatial-proximal digested ends of cross-linked DNA molecules;
(h) contacting the fourth spatial-proximal digested ends of cross-linked DNA molecules with ligase, thereby generating fourth cross-linked proximity-ligated DNA molecules comprising first, second, third and fourth ligation junctions;
(i) contacting the fourth cross-linked proximity-ligated DNA molecules comprising first, second, third and fourth ligation junctions with a reagent that reverses cross-linking, thereby generating proximity-ligated DNA molecules comprising first, second, third and fourth ligation junctions; and
(j) fragmenting the proximity-ligated DNA molecules to generate fragments of proximity-ligated DNA molecules comprising fragments spanning the first, second, third and fourth ligation junctions, wherein fragments spanning the first, second, third and fourth ligation junctions and of lengths that can be templates for short range sequencing, comprise sequences of essentially the whole genome or portion thereof.

B2. The method of embodiment B1, wherein the fragments spanning the first, second, third and fourth ligation junctions and of lengths that can be templates for short range sequencing comprise up 750 base pairs.

B3. The method of embodiments B1 or B2, wherein the first, second, third and fourth restriction endonucleases are selected from enzymes that generate molecules with ends having 5′ overhangs, 3′ overhangs or that are blunt and combinations thereof.

B4. The method of embodiment B3, wherein the first, second, third and fourth restriction endonucleases generate molecules with the same type of end.

B5. The method of embodiment B3, wherein two or more of the first, second, third and fourth restriction endonucleases generate molecules with different types of ends.

B5.1. The method of any one of embodiments B1 to B5, wherein one or more of the first, second, third and fourth restriction endonucleases require a specific buffer for high activity level different from a buffer required for high activity level by another of the first, second, third or fourth restriction endonucleases.

B5.2. The method of any one of embodiments B1 to B4, wherein the product of one or more of the first, second, third and fourth restriction endonucleases can incorporate a different label from the label incorporated by another of the first, second, third or fourth restriction endonucleases.

B6. The method of anyone of embodiments B1 to B5.2, wherein the DNA molecules are obtained from a sample selected from nuclei, cells, tissues, formalin-fixed paraffin-embedded (FFPE) samples, deeply formalin-fixed samples or cell-free DNA.

B7. The method of anyone of embodiments B1 to B5.4, wherein the DNA molecules are obtained from a single cell.

B7.1. The method of anyone of embodiments B1 to B5.4, wherein the DNA molecules are obtained from two or more cells.

B8. The method of any one of embodiments B1 to A5.4, wherein the cross-linked DNA molecules of a sample comprise two or more genomes or portions thereof.

B9. The method of anyone of embodiments B1 to B8, wherein the proximity-ligated DNA molecules are analyzed in a chromatin conformation assay.

B10. The method of embodiment B9, wherein the chromatin conformation assay is Capture-C, 3C, 4C, 5C, HiC, Capture-HiC, HiChIP, PLAC-seq, tethered chromosome capture (TCC), HiCulfite, Methyl-HiC, HiChIRP or combinations thereof.

B11. The method of embodiment B9, wherein the assay is genome-wide.

B11.1. The method of embodiment B11, wherein the assay is 3C, HiC, tethered chromosome capture (TCC), HiCulfite, Methyl-HiC or combinations thereof.

B12. The method of embodiment B9, wherein the assay is directed to one or more target regions in the genome.

B12.1. The method of embodiment B12, wherein the assays is Capture-C, 4C, 5C, Capture-HiC, HiChIP, PLAC-seq, HiChIRP or combinations thereof.

B13. The method of embodiment B12, wherein the targets are single nucleotide variations, insertions, deletions, copy number variations, genomic rearrangements or targets for phasing.

B14. The method embodiment B12 or B13, wherein the sample comprises a cancer genome and the target region is associated with a phenotype.

B15. The method of any one of embodiments B1 to B14, wherein the fragmented proximity-ligated DNA molecules are used to prepare a library of template molecules for DNA sequencing.

B16. The method of embodiment B15, wherein the fragmented proximity-ligated molecules are enriched for fragmented proximity-ligated DNA molecules comprising ligation junctions and the fragmented proximity-ligated DNA molecules comprising ligation junctions are used to prepare a library of template molecules for DNA sequencing.

B17. The method of embodiment B16, wherein the assay is HiC, Capture-HiC, HiSCIP, PLAC-seq, HiCulfite or Methyl-HiC and the ligation junctions are marked with an affinity purification marker.

B17.1. The method of embodiment B17, whereby enrichment is by affinity purification of the affinity purification marker with an affinity purification molecule.

B17.2. The method of embodiment B17.1, wherein the affinity purification molecule is streptavidin.

B17.3. The method of embodiment B16, where enrichment for fragmented proximity-ligated DNA molecules comprising ligation junctions is by size selection.

B18. The method of any one of embodiments B15 to B17.3, wherein the library of template molecules provides uniform genome-wide coverage of a genome or portion thereof.

B18.1. The method of any one of embodiments B15 to A18, wherein the library of template molecules is sequenced to generate sequence reads comprising sequence information.

B19. The method of embodiment B18.1, wherein the sequencing is short read sequencing.

B20. The method of embodiment B18.1 or B19, wherein the sequence information is utilized for genomic rearrangement analysis of the genome or portion thereof.

B21. The method of embodiment B20, wherein the genomic rearrangement analysis comprises identification of a breakpoint.

B22. The method of embodiment B21, wherein sequence information of a given sequence read is located upstream and downstream of the breakpoint.

B23. The method of embodiment B18.1 or B19, wherein the sequence information is utilized for clustering and ordering of contigs of the genome or portion thereof.

B24. The method of embodiment B23, wherein sequence information includes sequence information for each contig that is clustered and ordered.

B25. The method of embodiment B18.1 or B19, wherein the sequence information is utilized to determine contig orientation of the genome or portion thereof.

B26. The method of embodiment B18.1 or B19, wherein the sequence information is utilized for clustering, ordering and orientating contigs of the genome or portion thereof.

B27. The method of embodiment B18.1 or B19, wherein the sequence information is utilized for detection of pairwise 3D genome interactions of the genome or portion thereof.

B28. The method of embodiment B27, wherein the 3D genome interaction is between promoters, enhancers, gene regulatory elements, GWAS loci, chromatin loop and topological domain anchors, repetitive elements, polycomb regions, gene bodies, exons or integrated viral sequences.

B29. The method of embodiment B18.1 or B19, wherein the sequence information is utilized for protein factor location analysis and 3D conformation analysis of the genome or portion thereof.

B30. The method of embodiment B29, wherein the protein factor location analysis and 3D conformation analysis comprises PLAC-seq or HiChIP.

B31. The method of embodiment B18.1 or B19, wherein the sequence information is utilized for haplotype phasing of the genome or portion thereof.

B32. The method of embodiment B18.1 or B19, wherein the sequence information is utilized for genome assembly and 3D conformation analysis of the genome or portion thereof.

B33. The method of embodiment B18.1 or B19, wherein the sequence information is utilized for DNA methylation analysis of the genome or portion thereof.

B33.1. The method of embodiment B18.1 or B19, wherein the sequence information is utilized for DNA methylation analysis and detection of 3D genome interactions of the genome or portion thereof.

B34. The method of embodiment B18.1 or B19, wherein the sequence information is utilized for single nucleotide variant (SNV) discovery of the genome or portion thereof.

B35. The method of embodiment B18.1 or B19, wherein the sequence information is utilized for base polishing of long-range sequencing information of the genome or portion thereof.

B36. The method of embodiment B18.1 or B19, wherein the sequence information is utilized for highly sensitive copy number variation (CNV) analysis of the genome or portion thereof.

B37. The method of embodiment B36, wherein the copy number variation (CNV) is an amplification.

B38. The method of embodiment B36, wherein the copy number variation (CNV) is a heterozygous or homozygous deletion.

B39. The method of embodiment B18.1 or B19, wherein the sequence information is utilized for variant discovery, haplotype phasing and genome assembly of the genome or portion thereof.

B40. The method of embodiment B18.1 or B19, wherein the sequence information is utilized for haplotype phasing and genome assembly of the genome or portion thereof.

B41. The method of embodiment B18.1 or B19, wherein the sequence information is utilized for genome assembly and detection of 3D genome interaction of the genome or portion thereof.

C1. A method for preparing DNA molecules from a sample comprising:
(a) contacting cross-linked DNA molecules of a sample comprising a genome or portion thereof with a set of four restriction endonucleases; thereby generating spatial-proximal digested ends of cross-linked DNA molecules;
(b) contacting the spatial-proximal digested ends of cross-linked DNA molecules with one or more reagents that incorporate biotin-attached to a nucleotide into the spatially-proximal digested ends, thereby generating cross-linked DNA molecules comprising labelled spatially-proximal digested ends;
(c) contacting the cross-linked DNA molecules comprising labelled spatially-proximal digested ends with ligase, thereby generating cross-linked proximity-ligated DNA molecules comprising labelled ligation junctions;
(d) contacting cross-linked proximity-ligated DNA molecules comprising labelled ligation junctions with a reagent that reverses cross-linking, thereby generating proximity-ligated DNA molecules comprising labelled ligation junctions;
(e) fragmenting the proximity-ligated DNA molecules comprising labelled ligation junctions to generate fragments of proximity-ligated DNA molecules comprising fragments spanning the labelled ligation junctions, wherein fragments spanning the ligation junctions and of lengths that can be templates for short range sequencing, comprise sequences of essentially the whole genome or portion thereof; and
(f) enriching for DNA fragments spanning the labelled ligation junctions by affinity purification of labelled ligation junctions using an affinity purification molecule comprising streptavidin.

C2. The method of embodiment C1, wherein the fragments spanning the ligation junctions comprise fragments up 750 base pairs.

C3. The method of embodiment C1 or C2, where the streptavidin comprises streptavidin coated beads.

C4. The method of any one of embodiments C1 to C3, wherein each restriction endonuclease of the set has a high activity level in a common buffer and each restriction endonuclease of the set has a theoretical digestion frequency of at least 1 in 256.

C5. The method of any one of embodiments C1 to C4, wherein the restriction endonucleases are: MboI, HinfI, MseI and DdeI.

C5.1. The method of any one of embodiments C1 to C4, wherein the restriction endonucleases are: HpyCH4IV, HinfI, HinP1I and MseI.

C6. The method of anyone of embodiments C1 to C5.1, wherein the DNA molecules are obtained from a sample selected from nuclei, cells, tissues, formalin-fixed paraffin-embedded (FFPE) samples, deeply formalin-fixed samples or cell-free DNA.

C7. The method of anyone of embodiments C1 to C5.1, wherein the DNA molecules are obtained from a single cell.

C7.1. The method of anyone of embodiments C1 to C5.1, wherein the DNA molecules are obtained from two or more cells.

C8. The method of any one of embodiments C1 to C5.1, wherein the cross-linked DNA molecules of a sample comprise two or more genomes or portions thereof.

C9. The method of anyone of embodiments C1 to C8, wherein the proximity-ligated DNA molecules are analyzed in a chromatin conformation assay.

C10. The method of embodiment C9, wherein the chromatin conformation assay is Capture-C, 3C, 4C, 5C, HiC, Capture-HiC, HiChIP, PLAC-seq, tethered chromosome capture (TCC), HiCulfite, Methyl-HiC, HiChIRP or combinations thereof.

C11. The method of embodiment C9, wherein the assay is genome-wide.

C11.1. The method of embodiment C11, wherein the assay is 3C, HiC, tethered chromosome capture (TCC), HiCulfite, Methyl-HiC or combinations thereof.

C12. The method of embodiment C9, wherein the assay is directed to one or more target regions in the genome.

C12.1. The method of embodiment C12, wherein the assays is Capture-C, 4C, 5C, Capture-HiC, HiChIP, PLAC-seq, HiChIRP or combinations thereof.

C13. The method of embodiment C12, wherein the targets are single nucleotide variations, insertions, deletions, copy number variations, genomic rearrangements or targets for phasing.

C14. The method embodiment C12 or C13, wherein the sample comprises a cancer genome and the target region is associated with a phenotype.

C15. The method of any one of embodiments C1 to C14, wherein the fragmented proximity-ligated DNA molecules are used to prepare a library of template molecules for DNA sequencing.

C16. The method of embodiment C15, wherein the fragmented proximity-ligated molecules are enriched for fragmented proximity-ligated DNA molecules comprising ligation junctions and the fragmented proximity-ligated DNA molecules comprising ligation junctions are used to prepare a library of template molecules for DNA sequencing.

C17. The method of embodiment C16, wherein the assay is HiC, Capture-HiC, HiSCIP, PLAC-seq, HiCulfite or Methyl-HiC and the ligation junctions are marked with an affinity purification marker.

C17.1. The method of embodiment C17, whereby enrichment is by affinity purification of the affinity purification marker with an affinity purification molecule.

C17.2. The method of embodiment C17.1, wherein the affinity purification molecule is streptavidin.

C17.3. The method of embodiment C16, where enrichment for fragmented proximity-ligated DNA molecules comprising ligation junctions is by size selection.

C18. The method of any one of embodiments C15 to C17.3, wherein the library of template molecules provides uniform genome-wide coverage of a genome or portion thereof.

C18.1. The method of any one of embodiments C15 to C18, wherein the library of template molecules is sequenced to generate sequence reads comprising sequence information.

C19. The method of embodiment C18.1, wherein the sequencing is short read sequencing.

C20. The method of embodiment C18.1 or C19, wherein the sequence information is utilized for genomic rearrangement analysis of the genome or portion thereof.

C21. The method of embodiment C20, wherein the genomic rearrangement analysis comprises identification of a breakpoint.

C22. The method of embodiment C21, wherein sequence information of a given sequence read is located upstream and downstream of the breakpoint.

C23. The method of embodiment C18.1 or C19, wherein the sequence information is utilized for clustering and ordering of contigs of the genome or portion thereof.

C24. The method of embodiment C23, wherein sequence information includes sequence information for each contig that is clustered and ordered.

C25. The method of embodiment C18.1 or C19, wherein the sequence information is utilized to determine contig orientation of the genome or portion thereof.

C26. The method of embodiment C18.1 or C19, wherein the sequence information is utilized for clustering, ordering and orientating contigs of the genome or portion thereof.

C27. The method of embodiment C18.1 or C19, wherein the sequence information is utilized for detection of pairwise 3D genome interactions of the genome or portion thereof.

C28. The method of embodiment C27, wherein the 3D genome interaction is between promoters, enhancers, gene regulatory elements, GWAS loci, chromatin loop and topological domain anchors, repetitive elements, polycomb regions, gene bodies, exons or integrated viral sequences.

C29. The method of embodiment C18.1 or C19, wherein the sequence information is utilized for protein factor location analysis and 3D conformation analysis of the genome or portion thereof.

C30. The method of embodiment C29, wherein the protein factor location analysis and 3D conformation analysis comprises PLAC-seq or HiChIP.

C31. The method of embodiment C18.1 or C19, wherein the sequence information is utilized for haplotype phasing of the genome or portion thereof.

C32. The method of embodiment C18.1 or C19, wherein the sequence information is utilized for genome assembly and 3D conformation analysis of the genome or portion thereof.

C33. The method of embodiment C18.1 or C19, wherein the sequence information is utilized for DNA methylation analysis of the genome or portion thereof.

C33.1. The method of embodiment C18.1 or C19, wherein the sequence information is utilized for DNA methylation analysis and detection of 3D genome interactions of the genome or portion thereof.

C34. The method of embodiment C18.1 or C19, wherein the sequence information is utilized for single nucleotide variant (SNV) discovery of the genome or portion thereof.

C35. The method of embodiment C18.1 or C19, wherein the sequence information is utilized for base polishing of long-range sequencing information of the genome or portion thereof.

C36. The method of embodiment C18.1 or C19, wherein the sequence information is utilized for highly sensitive copy number variation (CNV) analysis of the genome or portion thereof.

C37. The method of embodiment C36, wherein the copy number variation (CNV) is an amplification.

C38. The method of embodiment C36, wherein the copy number variation (CNV) is a heterozygous or homozygous deletion.

C39. The method of embodiment C18.1 or C19, wherein the sequence information is utilized for variant discovery, haplotype phasing and genome assembly of the genome or portion thereof.

C40. The method of embodiment C18.1 or C19, wherein the sequence information is utilized for haplotype phasing and genome assembly of the genome or portion thereof.

C41. The method of embodiment C18.1 or C19, wherein the sequence information is utilized for genome assembly and detection of 3D genome interaction of the genome or portion thereof.

D1. A kit comprising:
(a) three or more restriction endonucleases;
(b) a restriction endonuclease buffer; and
(c) one or more of a biotinylated nucleotide, unlabeled nucleotides, a DNA polymerase, ligase, ligase buffer, one or more additional buffers and reagents for reversing cross-linking.

D2. The kit of embodiment D1, wherein the restriction endonucleases are in separate containers.

D3. The kit of embodiment D1, wherein the restriction endonucleases are in a single container.

D4. The kit of any one of embodiments D1 to D3, wherein each restriction endonuclease has a high activity level in a common restriction endonuclease buffer and each restriction endonuclease has a theoretical digestion frequency of at least 1 in 256.

D5. The kit of any one of embodiments D1 to D4, wherein the restriction endonuclease buffer is in a separate container from the restriction endonucleases.

D6. The kit of any one of embodiments D1 to D5, further comprising instructions.

E1. A kit comprising:
(a) four restriction endonucleases;
(b) a restriction endonuclease buffer; and
(c) one or more of a biotinylated nucleotide, unlabeled nucleotides, a DNA polymerase, ligase, ligase buffer one or more additional buffers and reagents for reversing cross-linking.

E2. The kit of embodiment E1, wherein the four restriction endonucleases are in separate containers.

E3. The kit of embodiment E1, wherein the four restriction endonucleases are in a single container.

E4. The kit of any one of embodiments E1 to E3, wherein the restriction endonuclease buffer is in a separate container from the four restriction endonucleases.

E5. The kit of any one of embodiments E1 to E4, wherein each restriction endonuclease has a high activity level in a common restriction endonuclease buffer and each restriction endonuclease has a theoretical digestion frequency of at least 1 in 256.

E6. The kit of any one of embodiments E1 to E5, wherein the four restriction endonucleases are: MboI, HinfI, MseI and DdeI.

E7. The kit of any one of embodiments E1 to E5, wherein the four restriction endonucleases are: HpyCH4IV, HinfI, HinP1I and MseI.

E8. The kit of any one of embodiments E1 to E7, further comprising instructions.

F1. A kit comprising:
(a) four restriction endonucleases;
(b) two or more restriction endonuclease buffers; and
(c) one or more of a biotinylated nucleotide, unlabeled nucleotides, a DNA polymerase, ligase, ligase buffer, one or more additional buffers and reagents for reversing cross-linking.

F2. The kit of embodiment F1, wherein the four restriction endonucleases are in separate containers.

F3. The kit of any one of embodiments F1 to F3, wherein the two or more restriction endonuclease buffers are in separate containers from the four restriction endonucleases.

F4. The kit of any one of embodiments F1 to F3, wherein each restriction endonuclease has a theoretical digestion frequency of at least 1 in 256.

F5. The kit of any one of embodiments F1 to F4, wherein at least two of the restriction endonucleases require unique buffers for high level activity.

F6. The kit of any one of embodiments F1 to F5, further comprising instructions.

G1. A method for preparing DNA molecules from a sample comprising:
(a) contacting spatially-proximal DNA molecules with stable spatial interactions from a sample, with two or more restriction endonucleases, thereby digesting the DNA molecules and generating spatial-proximal digested ends of DNA molecules; and
(b) contacting the spatial-proximal digested ends of DNA molecules with ligase, thereby generating proximity-ligated DNA molecules comprising ligation junctions, wherein the ligation junctions are unmarked.

G2. The method of embodiment G1, wherein the spatially-proximal DNA molecules comprise crosslinked DNA molecules.

G2.1. The method of embodiment G1 or G2, wherein the spatially-proximal DNA molecules with stable spatial interactions of a sample are within cells/nuclei and the contacting steps are in situ.

G2.2. The method of embodiment G1 or G2, wherein the spatially-proximal DNA molecules comprise a genome or portion thereof.

G3. The method of any one of embodiments G1 to G2.2, wherein there are two restriction endonucleases.

G4. The method of any one of embodiments G1 to G2.2, wherein there are at least three restriction endonucleases.

G4.1. The method of embodiment G4, wherein there are three restriction endonucleases.

G5. The method of any one of embodiments G1 to G4.1, wherein one of the restriction endonucleases is NlaIII.

G6. The method of any one of embodiments G1 to G5, wherein one of the restriction endonucleases is NlaIII and the other restriction endonuclease is MboI or MseI.

G7. The method of any one of embodiments G1 to G4.1, wherein one of the restriction endonucleases is NlaIII and another other restriction endonuclease is MboI or MseI.

G8. The method of embodiment G4 or G4.1, wherein the restriction endonucleases are: NlaIII, MboI and MseI.

G9. The method of any one of embodiments G1 to G5, wherein the restriction endonucleases produce the same overhanging sequence.

G10. The method of any one of embodiments G1 to G8, wherein the restriction endonucleases produce different overhanging sequences.

G11. The method of anyone of embodiments G1 to G10, wherein contact and digestion with all of the restriction endonucleases is at one time.

G12. The method of anyone of embodiments G1 to G10, wherein contact and digestion with each restriction endonucleases is sequential.

G12.1. The method of embodiment G12, wherein the digestion with a prior endonuclease or endonucleases has essentially completed.

G12.2. The method of embodiment G12, wherein the digestion with a prior endonuclease or endonucleases has not completed.

G13. The method of any one of embodiments G4 to G10, wherein contact and digestion with restriction endonucleases is sequential and at least one contact and digestion is with at least two restriction endonucleases.

G14. The method of any one of embodiments G12 to G13, wherein the sequential contact and digestion has a determined order for the restriction endonucleases.

G14.1. The method of embodiment G11, wherein contact with ligase is after completion of the digestion by the restriction endonucleases.

G14.2. The method of any one of embodiments G12 to G14, wherein contact with ligase is after completion of the sequential contact and digestion with all the restriction endonucleases.

G15. The method of any one of embodiments G12 to G14, wherein each contact and digestion with one or more restriction endonucleases is followed by contact with ligase.

G16. The method of anyone of embodiments G1 to G15, wherein the DNA molecules are obtained from a sample selected from nuclei, cells, tissues, formalin-fixed paraffin-embedded (FFPE) samples, deeply formalin-fixed samples or cell-free DNA.

G16.1 The method of embodiment G16, wherein the sample is in an aqueous solution or affixed to a solid surface.

G17. The method of anyone of embodiments G1 to G16.1, wherein the DNA molecules are obtained from a single cell.

G18. The method of anyone of embodiments G1 to G16.1, wherein the DNA molecules are obtained from two or more cells.

G19. The method of any one of embodiments G1 to G18, wherein the DNA molecules of a sample comprise two or more genomes or portions thereof.

G20. The method of any one of embodiments G1 to G19, wherein the method comprises one or more steps specific to a 4C, 5C, Capture-C, 3C-ChIP or Methyl-3C method.

G21. The method of any one of embodiments G1 to G20, wherein the proximity-ligated DNA molecules comprising ligation junctions are derived from sequences representing essentially an entire genome.

G22. The method of any one of embodiments G1 to G21, wherein the proximity-ligated DNA molecules comprising ligation junctions are purified.

G23. The method of any one of embodiments G2 to G22, wherein the crosslinked proximity-ligated DNA molecules comprising ligation junctions are contacted with a reagent that reverses crosslinking.

G24. The method of any one of embodiments, G1 to G23, wherein proximity-ligated DNA molecules comprising ligation junctions are enriched for DNA molecules with ligation junctions.

G24.1. The method of embodiment G24, wherein enrichment for DNA molecules with ligation junctions is by size selection.

G24.2. The method of embodiment G24.1, wherein size selection comprises the use of beads.

G24.3. The method of embodiment G24.1, wherein size selection comprises gel extraction or size selective DNA precipitation.

G25. The method of any one of embodiments G1 to G24.3, wherein a library of template molecules for DNA sequencing is prepared from the proximity-ligated DNA molecules.

G25.1. The method of embodiment G25, wherein size selection to enrich for DNA molecules with ligation junctions is performed before or after an amplification step when constructing the library.

G26. The method of embodiment G25 or G25.1, wherein the library of template molecules is sequenced to generate sequence reads comprising sequence information.

G27. The method of embodiment G26, wherein the sequencing is short-read sequencing.

G27.1. The method of any one of embodiments G1 to G27, wherein at least 30% of the nucleic acid templates are long-range cis molecules.

G27.2. The method of any one of embodiments G1 to G27, wherein at least 40% of the nucleic acid templates are long-range cis molecules.

G27.3. The method of any one of embodiments G1 to G27, wherein at least 50% of the nucleic acid templates are long-range cis molecules.

G27.4. The method of any one of embodiments G1 to G27, wherein at least 60% of the nucleic acid templates are long-range cis molecules.

G27.5. The method of embodiment G27, wherein the proximity-ligated DNA molecules are fragmented to generate fragments of proximity-ligated DNA molecules comprising fragments spanning the ligation junctions prior to the preparation of a library.

G27.6. The method of embodiment G26, wherein the sequencing is long-read sequencing.

G28. The method of any one of embodiments G26 to G27.6, wherein the sequence information is utilized for detection of pairwise 3D genome interactions of the genome or portion thereof.

G29. The method of embodiment G28, wherein the 3D genome interaction is between promoters, enhancers, gene regulatory elements, GWAS loci, chromatin loop and topological domain anchors, repetitive elements, polycomb regions, gene bodies, exons or integrated viral sequences.

G30. The method of any one of embodiments G26 to G27.6, wherein the sequence information is utilized for protein factor location analysis and 3D conformation analysis of the genome or portion thereof.

G31. The method of embodiment G30, wherein the protein factor location analysis and 3D conformation analysis comprises 3C-ChIP.

G32. The method of any one of embodiments G26 to G27.6, wherein the sequence information is utilized for genomic rearrangement analysis of the genome or portion thereof.

G33. The method of embodiment G32, wherein the genomic rearrangement analysis comprises identification of a breakpoint.

G34. The method of embodiment G33, wherein sequence information of a given sequence read is located upstream and downstream of the breakpoint.

G35. The method of any one of embodiments G26 to G27.6, wherein the sequence information is utilized for clustering and ordering of contigs of the genome or portion thereof.

G36. The method of embodiment G35, wherein sequence information includes sequence information for each contig that is clustered and ordered.

G37. The method of any one of embodiments G26 to G27.6, wherein the sequence information is utilized to determine contig orientation of the genome or portion thereof.

G38. The method of any one of embodiments G26 to G27.6, wherein the sequence information is utilized for clustering, ordering and orientating contigs of the genome or portion thereof.

G39. The method of any one of embodiments G26 to G27.6, wherein the sequence information is utilized for haplotype phasing of the genome or portion thereof.

G40. The method of any one of embodiments G26 to G27.6, wherein the sequence information is utilized for DNA methylation analysis of the genome or portion thereof.

G41. The method of any one of embodiments G26 to G27.6, wherein the sequence information is utilized for single nucleotide variant (SNV) discovery of the genome or portion thereof.

G42. The method of any one of embodiments G26 to G27.6, wherein the sequence information is utilized for base polishing of long-range sequencing information of the genome or portion thereof.

G43. The method of any one of embodiments G26 to G27.6, wherein the sequence information is utilized for highly sensitive copy number variation (CNV) analysis of the genome or portion thereof.

G44. The method of embodiment G43, wherein the copy number variation (CNV) is an amplification.

G45. The method of embodiment G43, wherein the copy number variation (CNV) is a heterozygous or homozygous deletion.

G46. The method of any one of embodiments G26 to G27.6, wherein the sequence information is utilized for variant discovery, haplotype phasing and genome assembly of the genome or portion thereof.

G47. The method of any one of embodiments G26 to G27.6, wherein the sequence information is utilized for variant discovery and haplotype phasing in a first sample comprising a paternal genome and a second sample comprising a maternal genome and the phased variants of the paternal genome and the maternal genome are used to analyze sequence data of a fetal genome obtained from cfDNA of the mother.

G48. The method of any one of embodiments G26 to G27.6, wherein the sequence information is utilized for haplotype phasing and genome assembly of the genome or portion thereof.

G49. The method of any one of embodiments G26 to G27.6, wherein the sequence information is utilized for genome assembly and 3D conformation analysis of the genome or portion thereof.

G50. The method of any one of embodiments G26 to G27.6, wherein the sequence information is utilized for DNA methylation analysis and detection of 3D genome interactions of the genome or portion thereof.

G51. The method of any one of embodiments G26 to G27.6, wherein the sequence information is utilized for genome assembly and detection of 3D genome interaction of the genome or portion thereof.

G52. The method of any one of embodiments G1 to G51, wherein molecular contiguity of proximity-ligated DNA molecules is preserved in barcodes.

G53. The method of embodiment G52, wherein barcodes are introduced into the proximity-ligated DNA molecules by contacting proximally-ligated DNA with a barcoded transposome linked bead prior to library preparation.

G54. The method of embodiment G52 to G53, wherein the sequence information is utilized for detection of higher-order 3D genome interactions of a genome or portion thereof, by leveraging the preserved molecular contiguity of proximity-ligated DNA molecules.

G55. The method of any one of embodiments G52 to G54, wherein the sequence information is utilized for detection of three or more concurrent 3D genome interactions of the genome or portion thereof, by leveraging the preserved molecular contiguity of proximity-ligated DNA molecules.

G56. The method of any one of embodiments G52 to G55, wherein the sequence information is utilized for detection of virtual pairwise 3D genome interactions by leveraging the preserved molecular contiguity of proximity-ligated DNA molecules.

G57. The method of embodiment G56, wherein a virtual pairwise 3D genome interaction is between restriction fragments that are not directly ligated to one another within a given proximity-ligated DNA molecule of the genome or portion thereof.

G58. The method of any one of embodiments G52 to G57, wherein the pairwise interactions, virtual pairwise interactions, and/or higher order interactions obtained by leveraging the preserved molecular contiguity of proximity ligated DNA molecules is utilized for 3D genome interactions of the genome or portion thereof, genomic rearrangement analysis of the genome or portion thereof, clustering and ordering of contigs of the genome or portion thereof, determining contig orientation of the genome or portion thereof, haplotype phasing of the genome or portion thereof, DNA methylation analysis of the genome or portion thereof, single nucleotide variant (SNV) discovery of the genome or portion thereof, base polishing of long-range sequencing information of the genome or portion thereof, highly sensitive copy number variation (CNV) analysis of the genome or portion thereof or combinations thereof.

H1. A method for preparing DNA molecules from a sample comprising:
(a) contacting spatially-proximal DNA molecules with stable spatial interactions from a sample, with a first restriction endonucleases, thereby digesting the DNA molecules and generating first spatial-proximal digested ends of DNA molecules;
(b) contacting the first spatial-proximal digested ends of DNA molecules with ligase, thereby generating first proximity-ligated DNA molecules comprising first ligation junctions, wherein the ligation junctions are unmarked;
(c) contacting the first proximity-ligated DNA molecules comprising first ligation junctions with a second restriction endonuclease, thereby digesting the first proximity-ligated DNA molecules and generating second spatial-proximal digested ends of DNA molecules; and
(d) contacting the second spatial-proximal digested ends of DNA molecules with ligase, thereby generating second proximity-ligated DNA molecules comprising first and second ligation junctions, wherein the ligation junctions are unmarked.

H2. The method of embodiment H1, comprising:
(e) the second proximity-ligated DNA molecules comprising first and second ligation junctions are contacted with a third restriction endonuclease, thereby digesting the second proximity-ligated DNA molecules and generating third spatial-proximal digested ends of DNA molecules; and
(f) contacting the third spatial-proximal digested ends of DNA molecules with ligase, thereby generating third proximity-ligated DNA molecules comprising first, second and third ligation junctions, wherein the ligation junctions are unmarked.

H3. A method for preparing DNA molecules from a sample comprising:
(a) contacting spatially-proximal DNA molecules with stable spatial interactions within cells/nuclei from a sample, with a first restriction endonucleases, thereby digesting the DNA molecules and generating first spatial-proximal digested ends of DNA molecules;
(b) contacting the first spatial-proximal digested ends of DNA molecules with ligase, thereby generating first proximity-ligated DNA molecules comprising first ligation junctions, wherein the ligation junctions are unmarked and the contacting steps are in situ;
(c) contacting the first proximity-ligated DNA molecules comprising first ligation junctions with a second restriction endonuclease, thereby digesting the first proximity-ligated DNA molecules and generating second spatial-proximal digested ends of DNA molecules; and
(d) contacting the second spatial-proximal digested ends of DNA molecules with ligase, thereby generating second proximity-ligated DNA molecules comprising first and second ligation junctions, wherein the ligation junctions are unmarked and the contacting steps are in situ.

H4. The method of embodiment H3, comprising:
(e) the second proximity-ligated DNA molecules comprising first and second ligation junctions are contacted with a third restriction endonuclease, thereby digesting the second proximity-ligated DNA molecules and generating third spatial-proximal digested ends of DNA molecules; and
(f) contacting the third spatial-proximal digested ends of DNA molecules with ligase, thereby generating third proximity-ligated DNA molecules comprising first, second and third ligation junctions, wherein the ligation junctions are unmarked and the contacting steps are in situ.

H5. The method of any one of embodiments H1 to H4, wherein the restriction endonucleases produce the same overhanging sequence.

H6. The method of any one of embodiments H1 to H4, wherein the restriction endonucleases produce different overhanging sequences.

11. A method of obtaining the spatial positioning of sequence information obtained from a proximity-ligated tissue section comprising:
(a) contacting a tissue section on a solid support comprising cells/nuclei having spatially-proximal DNA molecules with stable spatial interactions, with two or more restriction endonucleases, thereby digesting the DNA molecules and generating a tissue section with spatial-proximal digested ends of DNA molecules;
(b) contacting the spatial-proximal digested ends of DNA molecules of the tissue section with ligase, thereby generating a tissue section with proximity-ligated DNA molecules comprising ligation junctions, wherein the ligation junctions are unmarked or marked and the contacting steps are in situ.
(c) micro-dissecting the tissue section into spatially distinct regions;
(d) obtaining proximity-ligated DNA molecules from one or more spatially distinct regions;
(e) sequencing a library prepared using the proximity-ligated DNA molecules to generate sequence information; and
(f) assigning the sequence information from proximity-ligated molecules to a spatially distinct region of the tissue sample from which the proximity-ligated molecules were obtained, thereby obtaining the spatial positioning of sequence information.

12. A method of obtaining the spatial positioning of sequence information obtained from a proximity-ligated tissue section comprising:
(a) contacting a tissue section on a solid support comprising cells/nuclei having spatially-proximal DNA molecules with stable spatial interactions, with two or more restriction endonucleases, thereby digesting the DNA molecules and generating a tissue section with spatial-proximal digested ends of DNA molecules;
(b) contacting the spatial-proximal digested ends of DNA molecules of the tissue section with ligase, thereby generating a tissue section with proximity-ligated DNA molecules comprising ligation junctions, wherein the ligation junctions are unmarked or marked and the contacting steps are in situ.
(c) micro-dissecting the tissue section into spatially distinct regions;
(d) obtaining single cells comprising proximity-ligated DNA molecules from a spatially distinct region;
(e) sequencing a libraries prepared using the proximity-ligated DNA molecules of single cells to generate sequence information from single cells; and
(f) assigning the sequence information from single cells to a spatially distinct region of the tissue sample from which the cell was obtained, thereby obtaining the spatial positioning of sequence information from single cells.

13. A method of obtaining the spatial positioning of sequence information obtained from a proximity-ligated tissue section comprising:
(a) micro-dissecting a tissue section comprises cells/nuclei having spatially-proximal DNA molecules with stable spatial interactions into spatially distinct regions;
(b) contacting a spatially distinct region comprising cells/nuclei having spatially-proximal DNA molecules, with two or more restriction endonucleases, thereby digesting the DNA molecules and generating a spatially distinct region with spatial-proximal digested ends of DNA molecules;
(c) contacting the spatial-proximal digested ends of DNA molecules of the spatially distinct region with ligase, thereby generating a spatially distinct region with proximity-ligated DNA molecules comprising ligation junctions, wherein the ligation junctions are unmarked or marked and the contacting steps are in situ;
(d) obtaining proximity-ligated DNA molecules from one or more spatially distinct regions;
(e) sequencing a library prepared using the proximity-ligated DNA molecules from a spatially distinct region to generate sequence information; and
(f) assigning the sequence information from proximity-ligated molecules to a spatially distinct region of the tissue sample from which the proximity-ligated molecules were obtained, thereby obtaining the spatial positioning of sequence information.

14. A method of obtaining the spatial positioning of sequence information obtained from a proximity-ligated tissue section comprising:
(a) micro-dissecting a tissue section comprises cells/nuclei having spatially-proximal DNA molecules with stable spatial interactions into spatially distinct regions;
(b) contacting a spatially distinct region comprising cells/nuclei having spatially-proximal DNA molecules, with two or more restriction endonucleases, thereby digesting the DNA molecules and generating a spatially distinct region with spatial-proximal digested ends of DNA molecules;
(c) contacting the spatial-proximal digested ends of DNA molecules of the spatially distinct region with ligase, thereby generating a spatially distinct region with proximity-ligated DNA molecules comprising ligation junctions, wherein the ligation junctions are unmarked or marked and the contacting steps are in situ;
(d) obtaining single cells comprising proximity-ligated DNA molecules from a spatially distinct region;
(e) sequencing a libraries prepared using the proximity-ligated DNA molecules of single cells to generate sequence information from single cells; and
(f) assigning the sequence information from single cells to a spatially distinct region of the tissue sample from which the cell was obtained, thereby obtaining the spatial positioning of sequence information from single cells.

J1. A library of DNA template molecules for sequencing prepared by a method comprising any of the methods of embodiments A1 to A18.

J2. A library of DNA template molecules for sequencing prepared by a method comprising any of the methods of embodiments B1 to B14.

J3. A library of DNA template molecules for sequencing prepared by a method comprising any of the methods of embodiments C1 to C14.

J4. A library of DNA template molecules for sequencing prepared by a method comprising any of the methods of embodiments G1 to G27.5.

J5. A library of DNA template molecules for sequencing prepared by a method comprising any of the methods of embodiments H1 to H16.

K1. A kit comprising one or more of:
(a) two or more restriction endonucleases;
(b) a restriction endonuclease buffer; and
(c) one or more of unlabeled nucleotides, a DNA polymerase, a ligase, one or more additional buffers and reagents for reversing cross-linking, a Tn5 transposon, primers with barcode oligonucleotides, wherein the kit and does not include a biotinylated nucleotide or a labelled nucleotide.

K2. A kit comprising one or more of:
(a) two restriction endonucleases;
(b) a restriction endonuclease buffer; and
(c) one or more of unlabeled nucleotides, a DNA polymerase, a ligase, one or more additional buffers and reagents for reversing cross-linking, a Tn5 transposon, primers with barcode oligonucleotides, wherein the kit and does not include a biotinylated nucleotide or a labelled nucleotide.

K2.1. The kit of embodiment K2, wherein one of the restriction endonucleases is NlaIII.

K2.2. The kit of embodiment K2.1, wherein the other restriction endonuclease is MboI or MseI.

K3. A kit comprising one or more of:
(a) three restriction endonucleases;
(b) a restriction endonuclease buffer; and
(c) one or more of unlabeled nucleotides, a DNA polymerase, a ligase, one or more additional buffers and reagents for reversing cross-linking, a Tn5 transposon, primers with barcode oligonucleotides, wherein the kit and does not include a biotinylated nucleotide or a labelled nucleotide.

K3.1. The kit of embodiment K3, wherein one of the restriction endonucleases is NlaIII.

K3.2. The kit of embodiment K3.1, wherein one of the endonucleases is MboI or MseI.

K3.3. The kit of embodiment K3, wherein the restriction endonucleases are: NlaIII, MboI and MseI.

K4. The kit of any one of embodiments K1 to K3.3, wherein the restriction endonucleases of the kit produce the same overhanging sequence.

K5. The kit of any one of embodiments K1 to K3.3, wherein the restriction endonucleases of the kit produce different overhanging sequences.

K6. The kit of any one of embodiments K1 to K5, wherein digestion with the two or more restriction endonucleases of the kit can be carried out at the same time.

K7. The kit of any one of embodiments K1 to K5, wherein digestion with one or more restriction endonucleases of the kit cannot can be carried out at the same time.

K8. The kit of any one of embodiments K1 to K7, wherein the restriction endonucleases of the kit are in separate containers.

K9. The kit of embodiment K6, wherein the restriction endonucleases of the kit are in a single container.

K10. The kit of any one of embodiments K1 to K7, wherein the restriction endonucleases of the kit are in more than one container.

K10.1. The kit of embodiment K10, wherein at least one container contains more than one restriction endonuclease.

K11. The kit of any one of embodiments K1 to K6, wherein each restriction endonuclease of the kit has a high activity level in a common restriction endonuclease buffer and the buffer is in one container.

K12. The kit of any one of embodiments K1 to K10.1, wherein more than one restriction endonuclease buffer is in the kit and the buffers are in separate containers.

K13. The kit of any one of embodiments K1 to K12, wherein a restriction endonuclease buffer is in a separate container from a restriction endonuclease.

K14. The kit of any one of embodiments K1 to K13, wherein the kit comprises instructions.

K14.1. The kit of embodiment K14, wherein the instructions recite the order that the restriction enzymes of a kit are to be used.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for preparing DNA molecules from a sample comprising:
(a) contacting spatially-proximal DNA molecules with stable spatial interactions from a sample, with two or more restriction endonucleases, thereby digesting the DNA molecules and generating spatial-proximal digested ends of DNA molecules; and
(b) contacting the spatial-proximal digested ends of DNA molecules with ligase, thereby generating proximity-ligated DNA molecules comprising ligation junctions, wherein the ligation junctions are unmarked.

2. The method of claim 1, wherein the spatially-proximal DNA molecules comprise crosslinked DNA molecules.

3. The method of claim 1 or 2, wherein the spatially-proximal DNA molecules of a sample are within cells/nuclei and the contacting steps are in situ.

4. The method of claim 1 or 2, wherein the spatially-proximal DNA molecules comprise a genome or portion thereof.

5. The method of any one of claims 1 to 4, wherein there are two restriction endonucleases.

6. The method of any one of claims 1 to 4, wherein there are at least three restriction endonucleases.

7. The method of claim 6, wherein there are three restriction endonucleases.

8. The method of any one of claims 1 to 7, wherein one of the restriction endonucleases is NlaIII.

9. The method of any one of claims 1 to 5, wherein one of the restriction endonucleases is NlaIII and the other restriction endonuclease is MboI or MseI.

10. The method of any one of claims 1 to 7, wherein one of the restriction endonucleases is NlaIII and another restriction endonuclease is MboI or MseI.

11. The method of claim 6 or 7, wherein the restriction endonucleases are: NlaIII, MboI and MseI.

12. The method of any one of claims 1 to 7, wherein the restriction endonucleases produce the same overhanging sequence.

13. The method of any one of claims 1 to 11, wherein the restriction endonucleases produce different overhanging sequences.

14. The method of anyone of claims 1 to 13, wherein contact and digestion with all of the restriction endonucleases is at one time.

15. The method of anyone of claims 1 to 13 wherein contact and digestion with each restriction endonucleases is sequential.

16. The method of claim 15, wherein the digestion with a prior endonuclease or endonucleases has essentially completed.

17. The method of claim 15, wherein the digestion with a prior endonuclease or endonucleases has not completed.

18. The method of any one of claims 6 to 13, wherein contact and digestion with restriction endonucleases is sequential and at least one contact and digestion is with at least two restriction endonucleases.

19. The method of any one of claims 15 to 18, wherein the sequential contact and digestion has a determined order for the restriction endonucleases.

20. The method of claim 14, wherein contact with ligase is after completion of the digestion by the restriction endonucleases.

21. The method of any one of claims 15 to 19, wherein contact with ligase is after completion of the sequential contact and digestion with all the restriction endonucleases.

22. The method of any one of claims 15 to 19, wherein each contact and digestion with one or more restriction endonucleases is followed by contact with ligase.

23. The method of anyone of claims 1 to 22, wherein the DNA molecules are obtained from a sample selected from nuclei, cells, tissues, formalin-fixed paraffin-embedded (FFPE) samples, deeply formalin-fixed samples or cell-free DNA.

24. The method of claim 23, wherein the sample is in an aqueous solution or affixed to a solid surface.

25. The method of anyone of claims 1 to 24, wherein the DNA molecules are obtained from a single cell.

26. The method of anyone of claims 1 to 24, wherein the DNA molecules are obtained from two or more cells.

27. The method of any one of claims 1 to 26, wherein the DNA molecules of a sample comprise two or more genomes or portions thereof.

28. The method of any one of claims 1 to 27, wherein the method comprises one or more steps specific to a 4C, 5C, Capture-C, 3C-ChIP or Methyl-3C method.

29. The method of any one of claims 1 to 27, wherein the proximity-ligated DNA molecules comprising ligation junctions are derived from sequences representing essentially an entire genome.

30. The method of any one of claims 1 to 29, wherein the proximity-ligated DNA molecules comprising ligation junctions are purified.

31. The method of any one of claims 2 to 30, wherein the crosslinked proximity-ligated DNA molecules comprising ligation junctions are contacted with a reagent that reverses crosslinking.

32. The method of any one of claims, 1 to 31, wherein proximity-ligated DNA molecules comprising ligation junctions are enriched for DNA molecules with ligation junctions.

33. The method of claim 32, wherein enrichment for DNA molecules with ligation junctions is by size selection.

34. The method of claim 33, wherein size selection comprises the use of beads.

35. The method of claim 33, wherein size selection comprises gel extraction or size selective DNA precipitation.

36. The method of any one of claims 1 to 35, wherein a library of template molecules for DNA sequencing is prepared from the proximity-ligated DNA molecules.

37. The method of claim 36, wherein size selection to enrich for DNA molecules with ligation junctions is performed before or after an amplification step when constructing the library.

38. The method of claim 36 or 37, wherein the library of template molecules is sequenced to generate sequence reads comprising sequence information.

39. The method of claim 38, wherein the sequencing is short-read sequencing.

40. The method of any one of claims 1 to 39, wherein at least 30% of the nucleic acid templates are long-range cis molecules.

41. The method of any one of claims 1 to 39, wherein at least 40% of the nucleic acid templates are long-range cis molecules.

42. The method of any one of claims 1 to 39, wherein at least 50% of the nucleic acid templates are long-range cis molecules.

43. The method of any one of claims 1 to 39, wherein at least 60% of the nucleic acid templates are long-range cis molecules.

44. The method of claim 39, wherein the proximity-ligated DNA molecules are fragmented to generate fragments of proximity-ligated DNA molecules comprising fragments spanning the ligation junctions prior to the preparation of a library.

45. The method of claim 38, wherein the sequencing is long-read sequencing.

46. The method of any one of claims 38 to 45, wherein the sequence information is utilized for detection of pairwise 3D genome interactions of the genome or portion thereof.

47. The method of claim 46, wherein the 3D genome interaction is between promoters, enhancers, gene regulatory elements, GWAS loci, chromatin loop and topological domain anchors, repetitive elements, polycomb regions, gene bodies, exons or integrated viral sequences.

48. The method of any one of claims 38 to 45, wherein the sequence information is utilized for protein factor location analysis and 3D conformation analysis of the genome or portion thereof.

49. The method of claim 48, wherein the protein factor location analysis and 3D conformation analysis comprises 3C-ChIP.

50. The method of any one of claims 38 to 45, wherein the sequence information is utilized for genomic rearrangement analysis of the genome or portion thereof.

51. The method of claim 50, wherein the genomic rearrangement analysis comprises identification of a breakpoint.

52. The method of claim 51, wherein sequence information of a given sequence read is located upstream and downstream of the breakpoint.

53. The method of any one of claims 38 to 45, wherein the sequence information is utilized for clustering and ordering of contigs of the genome or portion thereof.

54. The method of claim 53, wherein sequence information includes sequence information for each contig that is clustered and ordered.

55. The method of any one of claims 38 to 45, wherein the sequence information is utilized to determine contig orientation of the genome or portion thereof.

56. The method of any one of claims 38 to 45, wherein the sequence information is utilized for clustering, ordering and orientating contigs of the genome or portion thereof.

57. The method of any one of claims 38 to 45, wherein the sequence information is utilized for haplotype phasing of the genome or portion thereof.

58. The method of any one of claims 38 to 45, wherein the sequence information is utilized for DNA methylation analysis of the genome or portion thereof.

59. The method of any one of claims 38 to 45, wherein the sequence information is utilized for single nucleotide variant (SNV) discovery of the genome or portion thereof.

60. The method of any one of claims 38 to 45, wherein the sequence information is utilized for base polishing of long-range sequencing information of the genome or portion thereof.

61. The method of any one of claims 38 to 45, wherein the sequence information is utilized for highly sensitive copy number variation (CNV) analysis of the genome or portion thereof.

62. The method of claim 61, wherein the copy number variation (CNV) is an amplification.

63. The method of claim 61, wherein the copy number variation (CNV) is a heterozygous or homozygous deletion.

64. The method of any one of claims 38 to 45, wherein the sequence information is utilized for variant discovery, haplotype phasing and genome assembly of the genome or portion thereof.

65. The method of any one of claims 38 to 45, wherein the sequence information is utilized for variant discovery and haplotype phasing in a first sample comprising a paternal genome and a second sample comprising a maternal genome and the phased variants of the paternal genome and the maternal genome are used to analyze sequence data of a fetal genome obtained from cfDNA of the mother.

66. The method of any one of claims 38 to 45, wherein the sequence information is utilized for haplotype phasing and genome assembly of the genome or portion thereof.

67. The method of any one of claims 38 to 45, wherein the sequence information is utilized for genome assembly and 3D conformation analysis of the genome or portion thereof.

68. The method of any one of claims 38 to 45, wherein the sequence information is utilized for DNA methylation analysis and detection of 3D genome interactions of the genome or portion thereof.

69. The method of any one of claims 38 to 45, wherein the sequence information is utilized for genome assembly and detection of 3D genome interaction of the genome or portion thereof.

70. The method of any one of claims 1 to 69, wherein molecular contiguity of proximity-ligated DNA molecules is preserved in barcodes.

71. The method of claim 70, wherein barcodes are introduced into the proximity-ligated DNA molecules by contacting proximally-ligated DNA with a barcoded transposome linked bead prior to library preparation.

72. The method of claim 70 or 71, wherein the sequence information is utilized for detection of higher-order 3D genome interactions of a genome or portion thereof, by leveraging the preserved molecular contiguity of proximity-ligated DNA molecules.

73. The method of any one of claims 70 to 72, wherein the sequence information is utilized for detection of three or more concurrent 3D genome interactions of the genome or portion thereof, by leveraging the preserved molecular contiguity of proximity-ligated DNA molecules.

74. The method of any one of claims 70 to 73, wherein the sequence information is utilized for detection of virtual pairwise 3D genome interactions by leveraging the preserved molecular contiguity of proximity-ligated DNA molecules.

75. The method of claim 74, wherein a virtual pairwise 3D genome interaction is between restriction fragments that are not directly ligated to one another within a given proximity-ligated DNA molecule of the genome or portion thereof.

76. The method of any one of claims 70 to 75, wherein the pairwise interactions, virtual pairwise interactions, and/or higher order interactions obtained by leveraging the preserved molecular contiguity of proximity ligated DNA molecules is utilized for 3D genome interactions of the genome or portion thereof, genomic rearrangement analysis of the genome or portion thereof, clustering and ordering of contigs of the genome or portion thereof, determining contig orientation of the genome or portion thereof, haplotype phasing of the genome or portion thereof, DNA methylation analysis of the genome or portion thereof, single nucleotide variant (SNV) discovery of the genome or portion thereof, base polishing of long-range sequencing information of the genome or portion thereof, highly sensitive copy number variation (CNV) analysis of the genome or portion thereof or combinations thereof.
